(19) United States
(12) Patent Application Publication
(10) Pub. No.: US 2022/0243220 A1
(43) Pub. Date: Aug. 4, 2022

(54) BIOTIC STRESS TOLERANT PLANTS AND METHODS

(71) Applicants: PIONEER OVERSEAS CORPORATION, JOHNSTON, IA (US); SINOBIOWAY BIO-AGRICULTURE GROUP CO. LTD., BEIJING (CN)

(72) Inventors: GUIHUA LU, SAN DIEGO, CA (US); GUOKUI WANG, BEIJING (CN); GUANFAN MAO, BEIJING (CN); RONGRONG JIAO, BEIJING (CN); AIFEN LIU, LAIYANG CITY (CN); FENG ZHONG, BEIJING (CN)

(73) Assignees: PIONEER OVERSEAS CORPORATION, JOHNSTON, IA (US); SINOBIOWAY BIO-AGRICULTURE GROUP CO. LTD., BEIJING (CN)

(21) Appl. No.: 17/596,376

(22) PCT Filed: Jul. 1, 2019
(86) PCT No.: PCT/CN2019/094186

§ 371 Date: Dec. 8, 2021

(51) Int. Cl. C12N 15/82; C07K 14/415; C12N 9/10; C12N 9/12

(52) U.S. Cl. CPC C12N 15/8286; C12N 9/12; C12N 9/1025; C07K 14/415

(57) ABSTRACT

The disclosure discloses isolated polynucleotides and polypeptides, and recombinant DNA constructs useful for conferring improved tolerance in plants to insect pests; compositions (such as plants or seeds) comprising these recombinant DNA constructs; and methods utilizing these recombinant DNA constructs. The recombinant DNA constructs comprise a polynucleotide operably linked to a promoter that is functional in a plant, wherein the polynucleotides encode insect tolerance polypeptides.

### FIELD

The field of the disclosure relates to plant breeding and genetics and, particularly, relates to improving insect pest tolerance in plants.

### BACKGROUND

Stresses to plants may be caused by both abiotic and biotic agents. For example, abiotic stresses include, for example, excessive or insufficient available water, temperature extremes, and synthetic chemicals such as herbicides. Biotic causes of stress include infection with pathogen, insect feeding, and parasitism by another plant such as mistletoe.

Pests' infestation can cause a huge financial loss annually either in crop loss or in purchasing expensive pesticides to keep check on pests. During the last centuries, synthetic chemical insecticidal compounds were used to control pests, while which poses many problems regarding the environment. Biotechnology in the last decades have presented new opportunities for pest control through genetic engineering. Advances in plant genetics coupled with the identification of insect growth factors and naturally-occurring plant defensive compounds or agents offer the opportunity to create transgenic crop plants capable of producing such defensive agents and thereby protect the plants against insect attack.

Certain species of microorganisms of the genus Bacillus are known to possess pesticidal activity against a range of insect pests including Lepidoptera, Diptera, Coleoptera, Hemiptera and others. Bacillus thuringiensis (Bt) and Bacillus popilliae are among the most successful biocontrol agents discovered to date. Insect pathogenicity has also been attributed to strains of B. larvae, B. lentimorbus, B. sphaericus and B. cereus. Microbial insecticides, particularly those obtained from Bacillus strains, have played an important role in agriculture as alternatives to chemical pest control.

Genetically engineered crops are now widely used in agriculture and have provided the farmer with an environmentally friendly and commercially attractive alternative to traditional insect control methods. While these genetically engineered, insect-resistant crop plants provide resistance to only a narrow range of the economically important insect pests. In some cases, insects can develop resistance to different insecticidal compounds, which raises the need to identify alternative biological control agents for pest control. Accordingly, there remains a need for new pesticidal proteins with different ranges of insecticidal activity against insect pests, e.g., insecticidal proteins which are active against a variety of insects in the order Lepidoptera and the order Coleoptera including but not limited to insect pests that have developed resistance to existing insecticides.

Accordingly, there is a need to develop compositions and methods that increase tolerance to insect pests in plants. This invention provides such compositions and methods.

### SUMMARY

The following embodiments are among those encompassed by the disclosure:

In one embodiment, the present disclosure includes an isolated polynucleotide, encoding a polypeptide with amino acid sequence of at least 90% sequence identity to SEQ ID NO: 3, 6, 9, 12, 15, 18, 21 or 24, wherein increased expression of the polynucleotide in a plant enhances insect tolerance. In certain embodiments, the isolated polynucleotide encodes the amino acid sequence of SEQ ID NO: 3, 6, 9, 12, 15, 18, 21 or 24. In certain embodiments, the isolated polynucleotide comprises the nucleotide sequence of SEQ ID NO: 1, 2, 4, 5, 7, 8, 10, 11, 13, 14, 16, 17, 19, 20, 22 or 23.

The present disclosure also provides a recombinant DNA construct comprising an isolated polynucleotide operably linked to at least one heterologous regulatory element, wherein the polynucleotide encodes a polypeptide with amino acid sequence of at least 90% sequence identity to SEQ ID NO: 3, 6, 9, 12, 15, 18, 21 or 24.

The present disclosure further provides a modified plant or seed having increased expression or activity of at least one polynucleotide encoding a polypeptide with amino acid sequence of at least 90% sequence identity to SEQ ID NO: 3, 6, 9, 12, 15, 18, 21 or 24. In certain embodiments, the modified plant or seed comprises in its genome a recombinant DNA construct comprising a polynucleotide operably linked to at least one heterologous regulatory element, wherein the polynucleotide encodes a polypeptide with amino acid sequence of at least 90% sequence identity to SEQ ID NO: 3, 6, 9, 12, 15, 18, 21 or 24. In certain embodiments, the modified plant exhibits improved insect tolerance compared to a control plant.

In certain embodiments, the modified plant or seed comprises a targeted genetic modification at a genomic locus comprising a polynucleotide encoding a polypeptide with amino acid sequence of at least 90% sequence identity to SEQ ID NO: 3, 6, 9, 12, 15, 18, 21 or 24, wherein the targeted genetic modification increase the expression and/or activity of the polypeptide. In certain embodiments, the modified plant exhibits improved insect tolerance compared to a control plant.

In certain embodiments, the insect tolerance is enhanced against any species of the orders selected from the group consisting of orders Coleoptera, Diptera, Hymenoptera, Lepidoptera, Mallophaga, Homoptera, Hemiptera Orthroptera, Thysanoptera, Dermaptera, Isoptera, Anoplura, Siphonaptera, Trichoptera, etc., particularly Lepidoptera. In certain embodiments, the insect pest is Asian Corn Borer (Ostrinia furnacalis), Rice Stem Borer (Chilo suppressalis), or Oriental Armyworm (Mythimna separata).

In certain embodiments, the plant is selected from the group consisting of rice, maize, soybean, sunflower, sorghum, canola, wheat, alfalfa, cotton, barley, millet, sugar cane and switchgrass.

Also provided are methods for increasing insect tolerance in a plant, the method comprising increasing the expression of at least one polynucleotide encoding a polypeptide with amino acid sequence of at least 90% sequence identity to SEQ ID NO: 3, 6, 9, 12, 15, 18, 21 or 24. Wherein the obtained plant exhibits increased insect tolerance when compared to the control plant.

In certain embodiments, the method for increasing insect tolerance comprises: (a) introducing into a regenerable plant cell a recombinant DNA construct comprising a polynucleotide operably linked to at least one heterologous regulatory element, wherein the polynucleotide encodes a polypeptide having an amino acid sequence of at least 80% sequence identity, when compared to SEQ ID NO: 3, 6, 9, 12, 15, 18, 21 or 24; and (b) generating the plant, wherein the plant comprises in its genome the recombinant DNA construct.

In certain embodiments, the method for increasing insect tolerance comprises: (a) introducing into a regenerable plant cell a targeted genetic modification at a genomic locus comprising a polynucleotide encoding a polypeptide having an amino acid sequence of at least 80% sequence identity, when compared to SEQ ID NO: 3, 6, 9, 12, 15, 18, 21 or 24; and (b) generating the plant, wherein the plant comprises in its genome the introduced genetic modification and has increased expression and/or activity of the polypeptide. In certain embodiments, the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), an engineered site-specific meganucleases, or an Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at 80% sequence identity, when compared to SEQ ID NO: 3, 6, 9, 12, 15, 18, 21 or 24.

### BRIEF DESCRIPTION OF THE DRAWINGS AND SEQUENCE LISTING

The disclosure can be more fully understood from the following detailed description and the accompanying Sequence Listing which form a part of this application. The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

TABLE 1
Sequence Listing Description

| Source | | SEQ ID NO: | SEQ ID NO: |
|---|---|---|---|
| species | Clone Designation | (Nucleotide) | (Amino Acid) |
| Oryza sativa | OsAAK1 | 1, 2 | 3 |
| Oryza sativa | OsDN-ITP8 | 4, 5 | 6 |
| Oryza sativa | OsPMR5 | 7, 8 | 9 |
| Oryza sativa | OsERV-B | 10, 11 | 12 |
| Oryza sativa | OsbHLH065 | 13, 14 | 15 |
| Oryza sativa | OsGRP1 | 16, 17 | 18 |
| Oryza sativa | OsAP2-4 | 19, 20 | 21 |
| Oryza sativa | OsDUF630/DUF632 | 22, 23 | 24 |
| Artificial | Primers | 25-40 | n/a |

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

### Definitions

As used herein, “increased insect tolerance” of a plant refers to a plant that inhibits the growth of, stunts the growth of, and/or kills one or more insect pests, including but not limited to, members of the Lepidoptera, Diptera, Hemiptera and Coleoptera orders as compared to a reference or control plant. Typically, when a plant comprising a recombinant DNA construct or DNA modification in its genome exhibits increased insect tolerance relative to a reference or control plant, the reference or control plant does not comprise in its genome the recombinant DNA construct or DNA modification.

“Pest” includes but is not limited to, insects, fungi, bacteria, nematodes, mites, ticks and the like. Insect pests include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Lepidoptera and Coleoptera.

Those skilled in the art will recognize that not all compounds are equally effective against all pests. Compounds of the embodiments display activity against insect pests, which may include economically important agronomic, forest, greenhouse, nursery ornamentals, food and fiber, public and animal health, domestic and commercial structure, household and stored product pests.

Larvae of the order Lepidoptera include, but are not limited to, armyworms, cutworms, loopers and heliothines in the family Noctuidae including Spodoptera frugiperda JE Smith (fall armyworm); S. exigua Hübner (beet armyworm); S. litura Fabricius (tobacco cutworm, cluster caterpillar); Mamestra configurata Walker (bertha armyworm); M. brassicae Linnaeus (cabbage moth); Agrotis ipsilon Hufnagel (black cutworm); A. orthogonia Morrison (western cutworm); A. subterranea Fabricius (granulate cutworm); Alabama argillacea Hübner (cotton leaf worm); Trichoplusia ni Hübner (cabbage looper); Pseudoplusia includens Walker (soybean looper); Anticarsia gemmatalis Hübner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); E. vittella Fabricius (spotted bollworm); Helicoverpa armigera Hübner (American bollworm); H. zea Boddie (corn earworm or cotton bollworm); Melanchra picta Harris (zebra caterpillar); Egira (Xylomyges) curialis Grote (citrus cutworm); Mythimna separate (Oriental Armyworm); borers, casebearers, webworms, coneworms, grass moths from the family Crambidae including Ostrinia furnacalis (Asian Corn Borer) and Ostrinia nubilalis (European Corn Borer), and skeletonizers from the family Pyralidae Ostrinia nubilalis Hübner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenee (rice leaf roller); Desmia funeralis Hübner (grape leaffolder); Diaphania hyalinata Linnaeus (melon worm); D. nitidalis Stoll (pickleworm); Diatraea grandiosella Dyar (southwestern corn borer), D. saccharalis Fabricius (surgarcane borer); Eoreuma loftini Dyar (Mexican rice borer); Ephestia elutella Hübner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hübner (Indian meal moth); Scirpophaga incertulas Walker (yellow stem borer); Udea rubigalis Guenee (celery leaftier); and leafrollers, budworms, seed worms and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rösslerstamm (summer fruit tortrix moth); Cochylis hospes Walsingham (banded sunflower moth); Cydia latiferreana Walsingham (filbertworm); C. pomonella Linnaeus (coding moth); Platynota flavedana Clemens (variegated leafroller); P. stultana Walsingham (omnivorous leafroller); Lobesia botrana Denis & Schiffermuller (European grape vine moth); Spilonota ocellana Denis & Schiffermuller (eyespotted bud moth); Endopiza viteana Clemens (grape berry moth); Eupoecilia ambiguella Hübner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp.

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guerin-Meneville (Chinese Oak Tussah Moth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Euproctis chrysorrhoea Linnaeus (browntail moth); Harrisina americana Guerin-Meneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vernata Peck (spring cankerworm); Papilio cresphontes Cramer (giant swallowtail orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval and Leconte (Southern cabbageworm); Sabulodes aegrotata Guenee (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer); Yponomeuta padella Linnaeus (ermine moth); Heliothis subflexa Guenee; Malacosoma spp. and Orgyia spp.

Of interest are larvae and adults of the order Coleoptera including weevils from the families Anthribidae, Bruchidae and Curculionidae (including, but not limited to: Anthonomus grandis Boheman (boll weevil); Lissorhoptrus oryzophilus Kuschel (rice water weevil); Sitophilus granarius Linnaeus (granary weevil); S. oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug)); flea beetles, cucumber beetles, rootworms, leaf beetles, potato beetles and leafminers in the family Chrysomelidae (including, but not limited to: Leptinotarsa decemlineata Say (Colorado potato beetle); Diabrotica virgifera virgifera LeConte (western corn rootworm); D. barberi Smith and Lawrence (northern corn rootworm); D. undecimpunctata howardi Barber (southern corn rootworm); Chaetocnema pulicaria Melsheimer (corn flea beetle); Phyllotreta cruciferae Goeze (Crucifer flea beetle); Phyllotreta striolata (stripped flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna varivestis Mulsant (Mexican bean beetle)); chafers and other beetles from the family Scarabaeidae (including, but not limited to: Popillia japonica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); Ligyrus gibbosus De Geer (carrot beetle)); carpet beetles from the family Dermestidae; wireworms from the family Elateridae, Eleodes spp., Melanotus spp.; Conoderus spp.; Limonius spp.; Agriotes spp.; Ctenicera spp.; Aeolus spp.; bark beetles from the family Scolytidae and beetles from the family Tenebrionidae.

Adults and immatures of the order Diptera are of interest, including leafminers Agromyza parvicornis Loew (corn blotch leafminer); midges (including, but not limited to: Contarinia sorghicola Coquillett (sorghum midge); Mayetiola destructor Say (Hessian fly); Sitodiplosis mosellana Gehin (wheat midge); Neolasioptera murtfeldtiana Felt, (sunflower seed midge)); fruit flies (Tephritidae), Oscinella frit Linnaeus (fruit flies); maggots (including, but not limited to: Delia platura Meigen (seedcorn maggot); D. coarctata Fallen (wheat bulb fly) and other Delia spp., Meromyza americana Fitch (wheat stem maggot); Musca domestica Linnaeus (house flies); Fannia canicularis Linnaeus, F. femoralis Stein (lesser house flies); Stomoxys calcitrans Linnaeus (stable flies)); face flies, horn flies, blow flies, Chrysomya spp.; Phormia spp. and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds) and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are adults and nymphs of the orders Hemiptera and Homoptera such as, but not limited to, adelgids from the family Adelgidae, plant bugs from the family Miridae, cicadas from the family Cicadidae, leafhoppers, Empoasca spp.; Cicadella viridis (Linnaeus) from the family Cicadellidae, planthoppers from the families Cixiidae, Flatidae, Fulgoroidea, Issidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Asterolecanidae, Coccidae, Dactylopiidae, Diaspididae, Eriococcidae, Ortheziidae, Phoenicococcidae and Margarodidae, lace bugs from the family Tingidae, stink bugs from the family Pentatomidae, cinch bugs, Blissus spp.; and other seed bugs from the family Lygaeidae, spittlebugs from the family Cercopidae squash bugs from the family Coreidae and red bugs and cotton stainers from the family Pyrrhocoridae.

Agronomically important members from the order Homoptera further include, but are not limited to: Acyrthisiphon pisum Harris (pea aphid); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacorthum solani Kaltenbach (foxglove aphid); Chaetosiphon fragaefolii Cockerell (strawberry aphid); Diuraphis noxia Kurdjumov/Mordvilko (Russian wheat aphid); Dysaphis plantaginea Paaserini (rosy apple aphid); Eriosoma lanigerum Hausmann (woolly apple aphid); Brevicoryne brassicae Linnaeus (cabbage aphid); Hyalopterus pruni Geoffroy (mealy plum aphid); Lipaphis erysimi Kaltenbach (turnip aphid); Metopolophium dirrhodum Walker (cereal aphid); Macrosiphum euphorbiae Thomas (potato aphid); Myzus persicae Sulzer (peach-potato aphid, green peach aphid); Nasonovia ribisnigri Mosley (lettuce aphid); Pemphigus spp. (root aphids and gall aphids); Rhopalosiphum maidis Fitch (corn leaf aphid); R. padi Linnaeus (bird cherry-oat aphid); Schizaphis graminum Rondani (greenbug); Sipha flava Forbes (yellow sugarcane aphid); Sitobion avenae Fabricius (English grain aphid); Therioaphis maculata Buckton (spotted alfalfa aphid); Toxoptera aurantii Boyer de Fonscolombe (black citrus aphid) and T. citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly); B. argentifolii Bellows & Perring (silverleaf whitefly); Dialeurodes citri Ashmead (citrus whitefly); Trialeurodes abutiloneus (bandedwinged whitefly) and T. vaporariorum Westwood (greenhouse whitefly); Empoasca fabae Harris (potato leafhopper); Laodelphax striatellus Fallen (smaller brown planthopper); Macrolestes quadrilineatus Forbes (aster leafhopper); Nephotettix cinticeps Uhler (green leafhopper); N. nigropictus Stäl (rice leafhopper); Nilaparvata lugens Stäl (brown planthopper); Peregrinus maidis Ashmead (corn planthopper); Sogatella furcifera Horvath (white-backed planthopper); Sogatodes orizicola Muir (rice delphacid); Typhlocyba pomaria McAtee (white apple leafhopper); Erythroneoura spp. (grape leafhoppers); Magicicada septendecim Linnaeus (periodical cicada); Icerya purchasi Maskell (cottony cushion scale); Quadraspidiotus perniciosus Comstock (San Jose scale); Planococcus citri Risso (citrus mealybug); Pseudococcus spp. (other mealybug complex); Cacopsylla pyricola Foerster (pear psylla); Trioza diospyri Ashmead (persimmon psylla).

Agronomically important species of interest from the order Hemiptera include, but are not limited to: Acrosternum hilare Say (green stink bug); Anasa tristis De Geer (squash bug); Blissus leucopterus leucopterus Say (chinch bug); Corythuca gossypii Fabricius (cotton lace bug); Cyrtopeltis modesta Distant (tomato bug); Dysdercus suturellus Herrich-Schsffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper).

Furthermore, embodiments may be effective against Hemiptera such, Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp. and Cimicidae spp.

Also included are adults and larvae of the order Acari (mites) such as Aceria tosichella Keifer (wheat curl mite); Petrobia latens Müller (brown wheat mite); spider mites and red mites in the family Tetranychidae, Panonychus ulmi Koch (European red mite); Tetranychus urticae Koch (two spotted spider mite); (T. mcdanieli McGregor (McDaniel mite); T. cinnabarinus Boisduval (carmine spider mite); T. turkestani Ugarov & Nikolski (strawberry spider mite); flat mites in the family Tenuipalpidae, Brevipalpus lewisi McGregor (citrus flat mite); rust and bud mites in the family Eriophyidae and other foliar feeding mites and mites important in human and animal health, i.e., dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick) and scab and itch mites in the families' Psoroptidae, Pyemotidae and Sarcoptidae.

Insect pests of the order Thysanura are of interest, such as Lepisma saccharina Linnaeus (silverfish); Thermobia domestica Packard (firebrat).

Additional arthropod pests covered include: spiders in the order Araneae such as Loxosceles reclusa Gertsch and Mulaik (brown recluse spider) and the Latrodectus mactans Fabricius (black widow spider) and centipedes in the order Scutigeromorpha such as Scutigera coleoptrata Linnaeus (house centipede).

Insect pest of interest include the superfamily of stink bugs and other related insects including but not limited to species belonging to the family Pentatomidae (Nezara viridula, Halyomorpha halys, Piezodorus guildini, Euschistus servus, Acrosternum hilare, Euschistus heros, Euschistus tristigmus, Dichelops furcatus, Dichelops melacanthus, and Bagrada hilaris (Bagrada Bug)), the family Plataspidae (Megacopta cribraria—Bean plataspid) and the family Cydnidae (Scaptocoris castanea—Root stink bug) and Lepidoptera species including but not limited to: diamond-back moth, e.g., Helicoverpa zea Boddie; soybean looper, e.g., Pseudoplusia includens Walker and velvet bean caterpillar e.g., Anticarsia gemmatalis Hübner.

Nematodes include parasitic nematodes such as root-knot, cyst and lesion nematodes, including Heterodera spp., Meloidogyne spp. and Globodera spp.; particularly members of the cyst nematodes, including, but not limited to, Heterodera glycines (soybean cyst nematode); Heterodera schachtii (beet cyst nematode); Heterodera avenae (cereal cyst nematode) and Globodera rostochiensis and Globodera pailida (potato cyst nematodes). Lesion nematodes include Pratylenchus spp.

Methods for measuring pesticidal activity are well known in the art. See, for example, Czapla and Lang, (1990) J. Econ. Entomol. 83: 2480-2485; Andrews, et al., (1988) Biochem. J. 252:199-206; Marrone, et al., (1985) J. of Economic Entomology 78:290-293 and U.S. Pat. No. 5,743,477, all of which are herein incorporated by reference in their entirety. Generally, the protein is mixed and used in feeding assays. See, for example Marrone, et al., (1985) J. of Economic Entomology 78:290-293. Such assays can include contacting plants with one or more pests and determining the plant's ability to survive and/or cause the death of the pests.

As used herein, the term “pesticidal activity” is used to refer to activity of an organism or a substance (such as, for example, a protein), whether toxic or inhibitory, that can be measured by, but is not limited to, pest mortality, pest weight loss, pest repellency, pest growth stunting, and other behavioral and physical changes of a pest after feeding and exposure for an appropriate length of time. In this manner, pesticidal activity impacts at least one measurable parameter of pest fitness. Similarly, “insecticidal activity” may be used to refer to “pesticidal activity” when the pest is an insect pest. “Stunting” is intended to mean greater than 50% inhibition of growth as determined by weight. General procedures for monitoring insecticidal activity include addition of the experimental compound or organism to the diet source in an enclosed container. Assays for assessing insecticidal activity are well known in the art. See, e.g., U.S. Pat. Nos. 6,570,005 and 6,339,144; herein incorporated by reference in their entirety. The optimal developmental stage for testing for insecticidal activity is larvae or immature forms of an insect of interest. The insects may be reared in total darkness at about 20-30° C. and about 30%˜70% relative humidity. Bioassays may be performed as described in Czapla and Lang (1990) J. Econ. Entomol. 83(6):2480-2485. Methods of rearing insect larvae and performing bioassays are well known to one of ordinary skill in the art.

Toxic and inhibitory effects of insecticidal proteins include, but are not limited to, stunting of larval growth, killing eggs or larvae, reducing either adult or juvenile feeding on transgenic plants relative to that observed on wild-type, and inducing avoidance behavior in an insect as it relates to feeding, nesting, or breeding as described herein, insect resistance can be conferred to an organism by introducing a nucleotide sequence encoding an insecticidal protein or applying an insecticidal substance, which includes, but is not limited to, an insecticidal protein, to an organism (e.g., a plant or plant part thereof). As used herein, “controlling a pest population” or “controls a pest” refers to any effect on a pest that results in limiting the damage that the pest causes. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack or deterring the pests from eating the plant.

“Agronomic characteristic” is a measurable parameter including but not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor and seedling emergence under low temperature stress.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

A “control”, “control plant” or “control plant cell” or the like provides a reference point for measuring changes in phenotype of a subject plant or plant cell in which genetic alteration, such as transformation, has been affected as to a gene of interest. For example, a control plant may be a plant having the same genetic background as the subject plant except for the genetic alteration that resulted in the subject plant or cell.

“Plant” includes reference to whole plants, plant organs, plant tissues, seeds and plant cells and progeny of the same. Plant cells include, without limitation, cells from seeds, suspension cultures, embryos, meristematic regions, callus tissues, leaves, roots, shoots, gametophytes, sporophytes, pollen, and microspores.

“Progeny” comprises any subsequent generation of a plant.

“Modified plant” includes reference to a plant which comprises within its genome a heterologous polynucleotide or modified gene or promoter. For example, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, and “nucleic acid fragment” are used interchangeably and refer to a polymer of RNA or DNA that is single- or double-stranded, optionally containing synthetic, non-natural or altered nucleotide bases. Nucleotides (usually found in their 5-monophosphate form) are referred to by their single-letter designation as follows: “A” for adenylate or deoxyadenylate, “C” for cytidylate or deoxycytidylate, and “G” for guanylate or deoxyguanylate for RNA or DNA, respectively; “U” for uridylate; “T” for deoxythymidylate; “R” for purines (A or G); “Y” for pyrimidines (C or T); “K” for G or T; “H” for A or C or T; “I” for inosine; and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, and sulfation, gamma-carboxylation of glutamic acid residues, hydroxylation and ADP-ribosylation.

“Recombinant DNA construct” refers to a combination of nucleic acid fragments that are not normally found together in nature. Accordingly, a recombinant DNA construct may comprise regulatory elements and coding sequences that are derived from different sources, or regulatory elements and coding sequences derived from the same source, but arranged in a manner different than that normally found in nature.

“Regulatory elements” refer to nucleotide sequences located upstream (5′ non-coding sequences), within, or downstream (3′ non-coding sequences) of a coding sequence, and influencing the transcription, RNA processing or stability, or translation of the associated coding sequence. Regulatory elements may include, but are not limited to, promoters, translation leader sequences, introns, and poly-adenylation recognition sequences. The terms “regulatory sequence” and “regulatory element” and “regulatory region” are used interchangeably herein.

“Promoter” refers to a nucleic acid fragment capable of controlling transcription of another nucleic acid fragment. “Promoter functional in a plant” is a promoter capable of controlling transcription of genes in plant cells whether its origin is from a plant cell or not. “Tissue-specific promoter” and “tissue-preferred promoter” refers to a promoter that is expressed predominantly but not necessarily exclusively in one tissue or organ, but that may also be expressed in one specific cell or cell type. “Developmentally regulated promoter” is a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

As used herein “increased”, “increase”, or the like refers to any detectable increase in an experimental group (e.g., plant with a DNA modification described herein) as compared to a control group (e.g., wild-type plant that does not comprise the DNA modification). Accordingly, increased expression of a protein comprises any detectable increase in the total level of the protein in a sample and can be determined using routine methods in the art such as, for example, Western blotting and ELISA.

As used herein, “sequence identity” or “identity” in the context of two polynucleotides or polypeptide sequences refer to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” is calculated by determining the number of positions at which the identical nucleic acid base or amino acid residue occurs in both sequences to yield the number of matched positions, dividing the number of matched positions by the total number of positions in the window of comparison, and multiplying the result by 100.

Unless stated otherwise, multiple alignments of the sequences provided herein are performed using the Clustal V method of alignment (Higgins and Sharp. (1989) CABIOS. 5:151-153) with the default parameters (GAP PENALTY=10, GAP LENGTH PENALTY=10). Default parameters for pairwise alignments and calculation of percent identity of amino acid sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

### Compositions:

### A. Polynucleotides and Polypeptides

The present disclosure provides polynucleotides encoding the following polypeptides:

One aspect of the disclosure provides a polynucleotide encoding a polypeptide comprising an amino acid sequence that is at least 80% identical (e.g. 81%, 82%, 83%, 84%, 85%, 86%, 87%, 88%, 89%, 90%, 91%, 92%, 93%, 94%, 95%, 96%, 97%, 98%, 99%, or 100%) to the amino acid sequence of any one of SEQ ID NO: 3 (OsAAK1), SEQ ID NO: 6 (OsDN-ITP8), SEQ ID NO: 9 (OsPMR5), SEQ ID NO: 12 (OsERV-B), SEQ ID NO: 15 (OsbHLH065), SEQ ID NO: 18 (OsGRP1), SEQ ID NO: 21 (OsAP2-4) and SEQ ID NO: 24 (OsDUF630/DUF632).

“OsAAK1” refers to a rice polypeptide that confers insect tolerance phenotype when overexpressed. The OsAAK1 polypeptide (SEQ ID NO: 3) is encoded by the coding sequence (CDS) (SEQ ID NO: 2) or nucleotide sequence (SEQ ID NO: 1) at rice gene locus LOC_Os04g46460.2, which is annotated as “Amino acid kinase, putative, expressed” in TIGR. “AAK1 polypeptide” refers herein to the OsAAK1 polypeptide and its paralogs or homologs from other organisms.

“OsDN-ITP8” refers to a rice polypeptide that confers insect tolerance phenotype when overexpressed. The OsDN-ITP8 polypeptide (SEQ ID NO: 6) is encoded by the coding sequence (CDS) (SEQ ID NO: 5) or nucleotide sequence (SEQ ID NO: 4) at rice gene locus LOC_Os03g16320.1, which is annotated as “Expressed protein” in TIGR. “DN-ITP8 polypeptide” refers herein to the OsDN-ITP8 polypeptide and its paralogs and homologs from other organisms.

“OsPMR5” refers to a rice polypeptide that confers insect tolerance phenotype when overexpressed. The OsPMR5 polypeptide (SEQ ID NO: 9) is encoded by the coding sequence (CDS) (SEQ ID NO: 8) or nucleotide sequence (SEQ ID NO: 7) at rice gene locus LOC_Os12g01560.1, which is annotated as “PMR5, putative, expressed” in TIGR. “PMR5 polypeptide” refers herein to the OsPMR5 polypeptide and its paralogs and homologs from other organisms.

“OsERV-B” refers to a rice polypeptide that confers insect tolerance phenotype when overexpressed. The OsERV-B polypeptide (SEQ ID NO: 12) is encoded by the coding sequence (CDS) (SEQ ID NO: 11) or nucleotide sequence (SEQ ID NO: 10) at rice gene locus LOC_Os06g38450.1, which is annotated as “vignain precursor, putative, expressed” in TIGR and “Ervatamin-B” in NCBI. “ERV-B polypeptide” refers herein to the OsERV-B polypeptide and its paralogs and homologs from other organisms.

“OsbHLH065” refers to a rice polypeptide that confers insect tolerance phenotype when overexpressed. The OsbHLH065 polypeptide (SEQ ID NO: 15) is encoded by the coding sequence (CDS) (SEQ ID NO: 14) or nucleotide sequence (SEQ ID NO: 13) at rice gene locus LOC_Os04g41570.2, which is annotated as “ethylene-responsive protein related, putative, expressed” in TIGR and “transcription factor bHLH153” in NCBI. “bHLH065 polypeptide” refers herein to the OsbHLH065 polypeptide and its paralogs and homologs from other organisms.

“OsGRP1” refers to a rice polypeptide that confers insect tolerance phenotype when overexpressed. The OsGRP1 polypeptide (SEQ ID NO: 18) is encoded by the coding sequence (CDS) (SEQ ID NO: 17) or nucleotide sequence (SEQ ID NO: 16) at rice gene locus LOC_Os04g41580.1, which is annotated as “glycine-rich protein, putative, expressed” in TIGR. “GRP1 polypeptide” refers herein to the OsGRP1 polypeptide and its paralogs and homologs from other organisms.

“OsAP2-4” refers to a rice polypeptide that confers insect tolerance phenotype when overexpressed. The OsAP2-4 polypeptide (SEQ ID NO: 21) is encoded by the coding sequence (CDS) (SEQ ID NO: 20) or nucleotide sequence (SEQ ID NO: 19) at rice gene locus LOC_Os04g46440.1, which is annotated as “AP2 domain containing protein, expressed” in TIGR. “AP2-4 polypeptide” refers herein to the OsAP2-4 polypeptide and its paralogs and homologs from other organisms.

“OsDUF630/DUF632” refers to a rice polypeptide that confers insect tolerance phenotype when overexpressed. The OsDUF630/DUF632 polypeptide (SEQ ID NO: 24) is encoded by the coding sequence (CDS) (SEQ ID NO: 23) or nucleotide sequence (SEQ ID NO: 22) at rice gene locus LOC_Os02g07850.1, which is annotated as “DUF630/DUF632 domains containing protein, putative, expressed” in TIGR. “DUF630/DUF632 polypeptide” refers herein to the OsDUF630/DUF632 polypeptide and its paralogs and homologs from other organisms.

By “insecticidal protein” is used herein to refer to a polypeptide that has toxic activity against one or more insect pests, including, but not limited to, members of the Lepidoptera, Diptera, Hemiptera and Coleoptera orders or the Nematoda phylum or a protein that has homology to such a protein. Pesticidal proteins have been isolated from organisms including, for example, Bacillus sp., Pseudomonas sp., Photorhabdus sp., Xenorhabdus sp., Clostridium bifermentans and Paenibacillus popilliae. Pesticidal proteins include but are not limited to: insecticidal proteins from Pseudomonas sp. such as PSEE3174 (Monalysin; (2011) PLoS Pathogens 7:1-13); from Pseudomonas protegens strain CHAO and Pf-5 (previously fluorescens) (Pechy-Tarr, (2008) Environmental Microbiology 10:2368-2386; GenBank Accession No. EU400157); from Pseudomonas Taiwanensis (Liu, et al., (2010) J. Agric. Food Chem., 58:12343-12349) and from Pseudomonas pseudoalcligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxicology Journal, 3:101-118 and Morgan, et al., (2001) Applied and Envir. Micro. 67:2062-2069); U.S. Pat. Nos. 6,048,838, and 6,379,946; a PIP1 polypeptide of US publication number US2014008054; an AflP-1A and/or AflP-1B polypeptide of U.S. Ser. No. 13/800,233; a PHI4 polypeptide of U.S. Ser. No. 13/839,702; and δ-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51, Cry55, Cry56, Cry57, Cry58, Cry59, Cry60, Cry61, Cry62, Cry63, Cry64, Cry65, Cry66, Cry67, Cry68, Cry69, Cry70, Cry71 and Cry72 classes of δ-endotoxin genes and the B. thuringiensis cytolytic cyt1 and cyt2 genes. Members of these classes of B. thuringiensis insecticidal proteins include, but are not limited to Cry1Aa1 (Accession #AAA22353); Cry1Aa2 (Accession #Accession #AAA22552); Cry1Aa3 (Accession #BAA00257); Cry1Aa4 (Accession #CAA31886); Cry1Aa5 (Accession #BAA04468); Cry1Aa6 (Accession #AAA86265); Cry1Aa7 (Accession #AAD46139); Cry1Aa8 (Accession #I26149); Cry1Aa9 (Accession #BAA77213); Cry1Aa10 (Accession #AAD55382); Cry1Aa11 (Accession #CAA70856); Cry1Aa12 (Accession #AAP80146); Cry1Aa13 (Accession #AAM44305); Cry1Aa14 (Accession #AAP40639); Cry1Aa15 (Accession #AAY66993); Cry1Aa16 (Accession #HQ439776); Cry1Aa17 (Accession #HQ439788); Cry1Aa18 (Accession #HQ439790); Cry1Aa19 (Accession #HQ685121); Cry1Aa20 (Accession #JF340156); Cry1Aa21 (Accession #JN651496); Cry1Aa22 (Accession #KC158223); Cry1Ab1 (Accession #AAA22330); Cry1Ab2 (Accession #AAA22613); Cry1Ab3 (Accession #AAA22561); Cry1Ab4 (Accession #BAA00071); Cry1Ab5 (Accession #CAA28405); Cry1Ab6 (Accession #AAA22420); Cry1Ab7 (Accession #CAA31620); Cry1Ab8 (Accession #AAA22551); Cry1Ab9 (Accession #CAA38701); Cry1Ab10 (Accession #A29125); Cry1Ab11 (Accession #I12419); Cry1Ab12 (Accession #AAC64003); Cry1Ab13 (Accession #AAN76494); Cry1Ab14 (Accession #AAG16877); Cry1Ab15 (Accession #AAO13302); Cry1Ab16 (Accession #AAK55546); Cry1Ab17 (Accession #AAT46415); Cry1Ab18 (Accession #AAQ88259); Cry1Ab19 (Accession #AAW31761); Cry1Ab20 (Accession #ABB72460); Cry1Ab21 (Accession #ABS18384); Cry1Ab22 (Accession #ABW87320); Cry1Ab23 (Accession #HQ439777); Cry1Ab24 (Accession #HQ439778); Cry1Ab25 (Accession #HQ685122); Cry1Ab26 (Accession #HQ847729); Cry1Ab27 (Accession #JN135249); Cry1Ab28 (Accession #JN135250); Cry1Ab29 (Accession #JN135251); Cry1Ab30 (Accession #JN135252); Cry1Ab31 (Accession #JN135253); Cry1Ab32 (Accession #JN135254); Cry1Ab33 (Accession #AAS93798); Cry1Ab34 (Accession #KC156668); Cry1Ab-like (Accession #AAK14336); Cry1Ab-like (Accession #AAK14337); Cry1Ab-like (Accession #AAK14338); Cry1Ab-like (Accession #ABG88858); Cry1Ac1 (Accession #AAA22331); Cry1Ac2 (Accession #AAA22338); Cry1Ac3 (Accession #CAA38098); Cry1Ac4 (Accession #AAA73077); Cry1Ac5 (Accession #AAA22339); Cry1Ac6 (Accession #AAA86266); Cry1Ac7 (Accession #AAB46989); Cry1Ac8 (Accession #AAC44841); Cry1Ac9 (Accession #AAB49768); Cry1Ac10 (Accession #CAA05505); Cry1Ac11 (Accession #CAA10270); Cry1Ac12 (Accession #I12418); Cry1Ac13 (Accession #AAD38701); Cry1Ac14 (Accession #AAQ06607); Cry1Ac15 (Accession #AAN07788); Cry1Ac16 (Accession #AAU87037); Cry1Ac17 (Accession #AAX18704); Cry1Ac18 (Accession #AAY88347); Cry1Ac19 (Accession #ABD37053); Cry1Ac20 (Accession #ABB89046); Cry1Ac21 (Accession #AAY66992); Cry1Ac22 (Accession #ABZ01836); Cry1Ac23 (Accession #CAQ30431); Cry1Ac24 (Accession #ABL01535); Cry1Ac25 (Accession #FJ513324); Cry1Ac26 (Accession #FJ617446); Cry1Ac27 (Accession #FJ617447); Cry1Ac28 (Accession #ACM90319); Cry1Ac29 (Accession #DQ438941); Cry1Ac30 (Accession #GQ227507); Cry1Ac31 (Accession #GU446674); Cry1Ac32 (Accession #HM061081); Cry1Ac33 (Accession #GQ866913); Cry1Ac34 (Accession #HQ230364); Cry1Ac35 (Accession #JF340157); Cry1Ac36 (Accession #JN387137); Cry1Ac37 (Accession #JQ317685); Cry1Ad1 (Accession #AAA22340); Cry1Ad2 (Accession #CAA01880); Cry1Ae1 (Accession #AAA22410); Cry1Af1 (Accession #AAB82749); Cry1Ag1 (Accession #AAD46137); Cry1Ah1 (Accession #AAQ14326); Cry1Ah2 (Accession #ABB76664); Cry1Ah3 (Accession #HQ439779); Cry1Ai1 (Accession #AAO39719); Cry1Ai2 (Accession #HQ439780); Cry1A-like (Accession #AAK14339); Cry1Ba1 (Accession #CAA29898); Cry1Ba2 (Accession #CAA65003); Cry1Ba3 (Accession #AAK63251); Cry1Ba4 (Accession #AAK51084); Cry1Ba5 (Accession #ABO20894); Cry1Ba6 (Accession #ABL60921); Cry1Ba7 (Accession #HQ439781); Cry1Bb1 (Accession #AAA22344); Cry1Bb2 (Accession #HQ439782); Cry1Bc1 (Accession #CAA86568); Cry1Bd1 (Accession #AAD10292); Cry1Bd2 (Accession #AAM93496); Cry1Be1 (Accession #AAC32850); Cry1Be2 (Accession #AAQ52387); Cry1Be3 (Accession #ACV96720); Cry1Be4 (Accession #HM070026); Cry1Bf1 (Accession #CAC50778); Cry1Bf2 (Accession #AAQ52380); Cry1Bg1 (Accession #AAO39720); Cry1Bh1 (Accession #HQ589331); Cry1Bi1 (Accession #KC156700); Cry1Ca1 (Accession #CAA30396); Cry1Ca2 (Accession #CAA31951); Cry1Ca3 (Accession #AAA22343); Cry1Ca4 (Accession #CAA01886); Cry1Ca5 (Accession #CAA65457); Cry1Ca6 [1](Accession #AAF37224); Cry1Ca7 (Accession #AAG50438); Cry1Ca8 (Accession #AAM00264); Cry1Ca9 (Accession #AAL79362); Cry1Ca10 (Accession #AAN16462); Cry1Ca11 (Accession #AAX53094); Cry1Ca12 (Accession #HM070027); Cry1Ca13 (Accession #HQ412621); Cry1Ca14 (Accession #JN651493); Cry1Cb1 (Accession #M97880); Cry1Cb2 (Accession #AAG35409); Cry1Cb3 (Accession #ACD50894); Cry1Cb-like (Accession #AAX63901); Cry1Da1 (Accession #CAA38099); Cry1Da2 (Accession #I176415); Cry1Da3 (Accession #HQ439784); Cry1Db1 (Accession #CAA80234); Cry1Db2 (Accession #AAK48937); Cry1Dc1 (Accession #ABK35074); Cry1Ea1 (Accession #CAA37933); Cry1Ea2 (Accession #CAA39609); Cry1Ea3 (Accession #AAA22345); Cry1Ea4 (Accession #AAD04732); Cry1Ea5 (Accession #AI5535); Cry1Ea6 (Accession #AAL50330); Cry1Ea7 (Accession #AAW72936); Cry1Ea8 (Accession #ABX11258); Cry1Ea9 (Accession #HQ439785); Cry1Ea10 (Accession #ADR00398); Cry1Ea11 (Accession #JQ652456); Cry1Eb1 (Accession #AAA22346); Cry1Fa1 (Accession #AAA22348); Cry1Fa2 (Accession #AAA22347); Cry1Fa3 (Accession #HM070028); Cry1Fa4 (Accession #HM439638); Cry1Fb1 (Accession #CAA80235); Cry1Fb2 (Accession #BAA25298); Cry1Fb3 (Accession #AAF21767); Cry1Fb4 (Accession #AAC10641); Cry1Fb5 (Accession #AAO13295); Cry1Fb6 (Accession #ACD50892); Cry1Fb7 (Accession #ACD50893); Cry1Ga1 (Accession #CAA80233); Cry1Ga2 (Accession #CAA70506); Cry1Gb1 (Accession #AAD10291); Cry1Gb2 (Accession #AAO13756); Cry1Gc1 (Accession #AAQ52381); Cry1Ha1 (Accession #CAA80236); Cry1Hb1 (Accession #AAA79694); Cry1Hb2 (Accession #HQ439786); Cry1H-like (Accession #AAF01213); Cry1Ia1 (Accession #CAA44633); Cry 1Ia2 (Accession #AAA22354); Cry 1Ia3 (Accession #AAC36999); Cry1Ia4 (Accession #AAB00958); Cry1Ia5 (Accession #CAA70124); Cry1Ia6 (Accession #AAC26910); Cry1Ia7 (Accession #AAM73516); Cry 1Ia8 (Accession #AAK66742); Cry1Ia9 (Accession #AAQ08616); Cry1Ia10 (Accession #AAP86782); Cry1Ia11 (Accession #CAC85964); Cry1Ia12 (Accession #AAV53390); Cry1Ia13 (Accession #ABF83202); Cry1Ia14 (Accession #ACG63871); Cry1Ia15 (Accession #FJ617445); Cry1Ia16 (Accession #FJ617448); Cry1Ia17 (Accession #GU989199); Cry1Ia18 (Accession #ADK23801); Cry1Ia19 (Accession #HQ439787); Cry1Ia20 (Accession #JQ228426); Cry1Ia21 (Accession #JQ228424); Cry1Ia22 (Accession #JQ228427); Cry1Ia23 (Accession #JQ228428); Cry1Ia24 (Accession #JQ228429); Cry1Ia25 (Accession #JQ228430); Cry1Ia26 (Accession #JQ228431); Cry1Ia27 (Accession #JQ228432); Cry1Ia28 (Accession #JQ228433); Cry1Ia29 (Accession #JQ228434); Cry1Ia30 (Accession #JQ317686); Cry1Ia31 (Accession #JX944038); Cry1Ia32 (Accession #JX944039); Cry1Ia33 (Accession #JX944040); Cry1Ib1 (Accession #AAA82114); Cry1Ib2 (Accession #ABW88019); Cry1Ib3 (Accession #ACD75515); Cry1Ib4 (Accession #HM051227); Cry1Ib5 (Accession #HM070028); Cry1Ib6 (Accession #ADK38579); Cry1Ib7 (Accession #JN571740); Cry1Ib8 (Accession #JN675714); Cry1Ib9 (Accession #JN675715); Cry1Ib10 (Accession #JN675716); Cry1Ib11 (Accession #JQ228423); Cry1Ic1 (Accession #AAC62933); Cry1Ic2 (Accession #AAE71691); Cry1Id1 (Accession #AAD44366); Cry1Id2 (Accession #JQ228422); Cry1Ie1 (Accession #AAG43526); Cry1Ie2 (Accession #HM439636); Cry1Ie3 (Accession #KC156647); Cry1Ie4 (Accession #KC156681); Cry1If1 (Accession #AAQ52382); Cry1Ig1 (Accession #KC156701); Cry1I-like (Accession #AAC31094); Cry1I-like (Accession #ABG88859); Cry1Ja1 (Accession #AAA22341); Cry1Ja2 (Accession #HM070030); Cry1Ja3 (Accession #JQ228425); Cry1Jb1 (Accession #AAA98959); Cry1Jc1 (Accession #AAC31092); Cry1Jc2 (Accession #AAQ52372); Cry1Jd1 (Accession #CAC50779); Cry1Ka1 (Accession #AAB00376); Cry1Ka2 (Accession #HQ439783); Cry1La1 (Accession #AAS60191); Cry1La2 (Accession #HM070031); Cry1Ma1 (Accession #FJ884067); Cry1Ma2 (Accession #KC156659); Cry1Na1 (Accession #KC156648); Cry1Nb1 (Accession #KC156678); Cry1-like (Accession #AAC31091); Cry2Aa1 (Accession #AAA22335); Cry2Aa2 (Accession #AAA83516); Cry2Aa3 (Accession #D86064); Cry2Aa4 (Accession #AAC04867); Cry2Aa5 (Accession #CAA10671); Cry2Aa6 (Accession #CAA10672); Cry2Aa7 (Accession #CAA10670); Cry2Aa8 (Accession #AAO13734); Cry2Aa9 (Accession #AAO13750); Cry2Aa10 (Accession #AAQ04263); Cry2Aa11 (Accession #AAQ52384); Cry2Aa12 (Accession #ABI83671); Cry2Aa13 (Accession #ABL01536); Cry2Aa14 (Accession #ACF04939); Cry2Aa15 (Accession #JN426947); Cry2Ab1 (Accession #AAA22342); Cry2Ab2 (Accession #CAA39075); Cry2Ab3 (Accession #AAG36762); Cry2Ab4 (Accession #AAO13296); Cry2Ab5 (Accession #AAQ04609); Cry2Ab6 (Accession #AAP59457); Cry2Ab7 (Accession #AAZ66347); Cry2Ab8 (Accession #ABC95996); Cry2Ab9 (Accession #ABC74968); Cry2Ab10 (Accession #EF157306); Cry2Ab11 (Accession #CAM84575); Cry2Ab12 (Accession #ABM21764); Cry2Ab13 (Accession #ACG76120); Cry2Ab14 (Accession #ACG76121); Cry2Ab15 (Accession #HM037126); Cry2Ab16 (Accession #GQ866914); Cry2Ab17 (Accession #HQ439789); Cry2Ab18 (Accession #JN135255); Cry2Ab19 (Accession #JN135256); Cry2Ab20 (Accession #JN135257); Cry2Ab21 (Accession #JN135258); Cry2Ab22 (Accession #JN135259); Cry2Ab23 (Accession #JN135260); Cry2Ab24 (Accession #JN135261); Cry2Ab25 (Accession #JN415485); Cry2Ab26 (Accession #JN426946); Cry2Ab27 (Accession #JN415764); Cry2Ab28 (Accession #JN651494); Cry2Ac1 (Accession #CAA40536); Cry2Ac2 (Accession #AAG35410); Cry2Ac3 (Accession #AAQ52385); Cry2Ac4 (Accession #ABC95997); Cry2Ac5 (Accession #ABC74969); Cry2Ac6 (Accession #ABC74793); Cry2Ac7 (Accession #CAL18690); Cry2Ac8 (Accession #CAM09325); Cry2Ac9 (Accession #CAM09326); Cry2Ac10 (Accession #ABN15104); Cry2Ac11 (Accession #CAM83895); Cry2Ac12 (Accession #CAM83896); Cry2Ad1 (Accession #AAF09583); Cry2Ad2 (Accession #ABC86927); Cry2Ad3 (Accession #CAK29504); Cry2Ad4 (Accession #CAM32331); Cry2Ad5 (Accession #CAO78739); Cry2Ae1 (Accession #AAQ52362); Cry2Af1 (Accession #ABO30519); Cry2Af2 (Accession #GQ866915); Cry2Ag1 (Accession #ACH91610); Cry2Ah1 (Accession #EU939453); Cry2Ah2 (Accession #ACL80665); Cry2Ah3 (Accession #GU073380); Cry2Ah4 (Accession #KC156702); Cry2Ai1 (Accession #FJ788388); Cry2Aj (Accession #); Cry2Ak1 (Accession #KC156660); Cry2Ba1 (Accession #KC156658); Cry3Aa1 (Accession #AAA22336); Cry3Aa2 (Accession #AAA22541); Cry3Aa3 (Accession #CAA68482); Cry3Aa4 (Accession #AAA22542); Cry3Aa5 (Accession #AAA50255); Cry3Aa6 (Accession #AAC43266); Cry3Aa7 (Accession #CAB41411); Cry3Aa8 (Accession #AAS79487); Cry3Aa9 (Accession #AAW05659); Cry3Aa10 (Accession #AAU29411); Cry3Aa11 (Accession #AAW82872); Cry3Aa12 (Accession #ABY49136); Cry3Ba1 (Accession #CAA34983); Cry3Ba2 (Accession #CAA00645); Cry3Ba3 (Accession #JQ397327); Cry3Bb1 (Accession #AAA22334); Cry3Bb2 (Accession #AAA74198); Cry3Bb3 (Accession #I15475); Cry3Ca1 (Accession #CAA42469); Cry4Aa1 (Accession #CAA68485); Cry4Aa2 (Accession #BAA00179); Cry4Aa3 (Accession #CAD30148); Cry4Aa4 (Accession #AFB18317); Cry4A-like (Accession #AAY96321); Cry4Ba1 (Accession #CAA30312); Cry4Ba2 (Accession #CAA30114); Cry4Ba3 (Accession #AAA22337); Cry4Ba4 (Accession #BAA00178); Cry4Ba5 (Accession #CAD30095); Cry4Ba-like (Accession #ABC47686); Cry4Ca1 (Accession #EU646202); Cry4Cb1 (Accession #FJ403208); Cry4Cb2 (Accession #FJ597622); Cry4Cc1 (Accession #FJ403207); Cry5Aa1 (Accession #AAA67694); Cry5Ab1 (Accession #AAA67693); Cry5Ac1 (Accession #I34543); Cry5Ad1 (Accession #ABQ82087); Cry5Ba1 (Accession #AAA68598); Cry5Ba2 (Accession #ABW88931); Cry5Ba3 (Accession #AFJ04417); Cry5Ca1 (Accession #HM461869); Cry5Ca2 (Accession #ZP_04123426); Cry5Da1 (Accession #HM461870); Cry5Da2 (Accession #ZP_04123980); Cry5Ea1 (Accession #HM485580); Cry5Ea2 (Accession #ZP_04124038); Cry6Aa1 (Accession #AAA22357); Cry6Aa2 (Accession #AAM46849); Cry6Aa3 (Accession #ABH03377); Cry6Ba1 (Accession #AAA22358); Cry7Aa1 (Accession #AAA22351); Cry7Ab1 (Accession #AAA21120); Cry7Ab2 (Accession #AAA21121); Cry7Ab3 (Accession #ABX24522); Cry7Ab4 (Accession #EU380678); Cry7Ab5 (Accession #ABX79555); Cry7Ab6 (Accession #ACI44005); Cry7Ab7 (Accession #ADB89216); Cry7Ab8 (Accession #GU145299); Cry7Ab9 (Accession #ADD92572); Cry7Ba1 (Accession #ABB70817); Cry7Bb1 (Accession #KC156653); Cry7Ca1 (Accession #ABR67863); Cry7Cb1 (Accession #KC156698); Cry7Da1 (Accession #ACQ99547); Cry7Da2 (Accession #HM572236); Cry7Da3 (Accession #KC156679); Cry7Ea1 (Accession #HM035086); Cry7Ea2 (Accession #HM132124); Cry7Ea3 (Accession #EEM19403); Cry7Fa1 (Accession #HM035088); Cry7Fa2 (Accession #EEM19090); Cry7Fb1 (Accession #HM572235); Cry7Fb2 (Accession #KC156682); Cry7Ga1 (Accession #HM572237); Cry7Ga2 (Accession #KC156669); Cry7Gb1 (Accession #KC156650); Cry7Gc1 (Accession #KC156654); Cry7Gd1 (Accession #KC156697); Cry7Ha1 (Accession #KC156651); Cry7Ia1 (Accession #KC156665); Cry7Ja1 (Accession #KC156671); Cry7Ka1 (Accession #KC156680); Cry7Kb1 (Accession #BAM99306); Cry7La1 (Accession #BAM99307); Cry8Aa1 (Accession #AAA21117); Cry8Ab1 (Accession #EU044830); Cry8Ac1 (Accession #KC156662); Cry8Ad1 (Accession #KC156684); Cry8Ba1 (Accession #AAA21118); Cry8Bb1 (Accession #CAD57542); Cry8Bc1 (Accession #CAD57543); Cry8Ca1 (Accession #AAA21119); Cry8Ca2 (Accession #AAR98783); Cry8Ca3 (Accession #EU625349); Cry8Ca4 (Accession #ADB54826); Cry8Da1 (Accession #BAC07226); Cry8Da2 (Accession #BD133574); Cry8Da3 (Accession #BD133575); Cry8Db1 (Accession #BAF93483); Cry8Ea1 (Accession #AAQ73470); Cry8Ea2 (Accession #EU047597); Cry8Ea3 (Accession #KC855216); Cry8Fa1 (Accession #AAT48690); Cry8Fa2 (Accession #HQ174208); Cry8Fa3 (Accession #AFH78109); Cry8Ga1 (Accession #AAT46073); Cry8Ga2 (Accession #ABC42043); Cry8Ga3 (Accession #FJ198072); Cry8Ha1 (Accession #AAW81032); Cry8Ia1 (Accession #EU381044); Cry8Ia2 (Accession #GU073381); Cry8Ia3 (Accession #HM044664); Cry8Ia4 (Accession #KC156674); Cry8Ib1 (Accession #GU325772); Cry8Ib2 (Accession #KC156677); Cry8Ja1 (Accession #EU625348); Cry8Ka1 (Accession #FJ422558); Cry8Ka2 (Accession #ACN87262); Cry8Kb1 (Accession #HM123758); Cry8Kb2 (Accession #KC156675); Cry8La1 (Accession #GU325771); Cry8Ma1 (Accession #HM044665); Cry8Ma2 (Accession #EEM86551); Cry8Ma3 (Accession #HM210574); Cry8Na1 (Accession #HM640939); Cry8Pa1 (Accession #HQ388415); Cry8Qa1 (Accession #HQ441166); Cry8Qa2 (Accession #KC152468); Cry8Ra1 (Accession #AFP87548); Cry8Sa1 (Accession #JQ740599); Cry8Ta1 (Accession #KC156673); Cry8-like (Accession #FJ770571); Cry8-like (Accession #ABS53003); Cry9Aa1 (Accession #CAA41122); Cry9Aa2 (Accession #CAA41425); Cry9Aa3 (Accession #GQ249293); Cry9Aa4 (Accession #GQ249294); Cry9Aa5 (Accession #JX174110); Cry9Aa like (Accession #AAQ52376); Cry9Ba1 (Accession #CAA52927); Cry9Ba2 (Accession #GU299522); Cry9Bb1 (Accession #AAV28716); Cry9Ca1 (Accession #CAA85764); Cry9Ca2 (Accession #AAQ52375); Cry9Da1 (Accession #BAA19948); Cry9Da2 (Accession #AAB97923); Cry9Da3 (Accession #GQ249293); Cry9Da4 (Accession #GQ249297); Cry9Db1 (Accession #AAX78439); Cry9Dc1 (Accession #KC156683); Cry9Ea1 (Accession #BAA34908); Cry9Ea2 (Accession #AAO12908); Cry9Ea3 (Accession #ABM21765); Cry9Ea4 (Accession #ACE88267); Cry9Ea5 (Accession #ACF04743); Cry9Ea6 (Accession #ACG63872); Cry9Ea7 (Accession #FJ380927); Cry9Ea8 (Accession #GQ249292); Cry9Ea9 (Accession #JN651495); Cry9Eb1 (Accession #CAC50780); Cry9Eb2 (Accession #GQ249298); Cry9Eb3 (Accession #KC156646); Cry9Ec1 (Accession #AAC63366); Cry9Ed1 (Accession #AAX78440); Cry9Ee1 (Accession #GQ249296); Cry9Ee2 (Accession #KC156664); Cry9Fa1 (Accession #KC156692); Cry9Ga1 (Accession #KC156699); Cry9-like (Accession #AAC63366); Cry10Aa1 (Accession #AAA22614); Cry10Aa2 (Accession #E00614); Cry10Aa3 (Accession #CAD30098); Cry10Aa4 (Accession #AFB18318); Cry10A-like (Accession #DQ167578); Cry11Aa1 (Accession #AAA22352); Cry11Aa2 (Accession #AAA22611); Cry11Aa3 (Accession #CAD30081); Cry11Aa4 (Accession #AFB18319); Cry11Aa-like (Accession #DQ166531); Cry11Ba1 (Accession #CAA60504); Cry11Bb1 (Accession #AAC97162); Cry11Bb2 (Accession #HM068615); Cry12Aa1 (Accession #AAA22355); Cry13Aa1 (Accession #AAA22356); Cry14Aa1 (Accession #AAA21516); Cry14Ab1 (Accession #KC156652); Cry15Aa1 (Accession #AAA22333); Cry16Aa1 (Accession #CAA63860); Cry17Aa1 (Accession #CAA67841); Cry18Aa1 (Accession #CAA67506); Cry18Ba1 (Accession #AAF89667); Cry18Ca1 (Accession #AAF89668); Cry19Aa1 (Accession #CAA68875); Cry19Ba1 (Accession #BAA32397); Cry19Ca1 (Accession #AFM37572); Cry20Aa1 (Accession #AAB93476); Cry20Ba1 (Accession #ACS93601); Cry20Ba2 (Accession #KC156694); Cry20-like (Accession #GQ144333); Cry21Aa1 (Accession #I32932); Cry21Aa2 (Accession #I66477); Cry21Ba1 (Accession #BAC06484); Cry21Ca1 (Accession #JF521577); Cry21Ca2 (Accession #KC156687); Cry21Da1 (Accession #JF521578); Cry22Aa1 (Accession #I34547); Cry22Aa2 (Accession #CAD43579); Cry22Aa3 (Accession #ACD93211); Cry22Ab1 (Accession #AAK50456); Cry22Ab2 (Accession #CAD43577); Cry22Ba1 (Accession #CAD43578); Cry22Bb1 (Accession #KC156672); Cry23Aa1 (Accession #AAF76375); Cry24Aa1 (Accession #AAC61891); Cry24Ba1 (Accession #BAD32657); Cry24Ca1 (Accession #CAJ43600); Cry25Aa1 (Accession #AAC61892); Cry26Aa1 (Accession #AAD25075); Cry27Aa1 (Accession #BAA82796); Cry28Aa1 (Accession #AAD24189); Cry28Aa2 (Accession #AAG00235); Cry29Aa1 (Accession #CAC80985); Cry30Aa1 (Accession #CAC80986); Cry30Ba1 (Accession #BAD00052); Cry30Ca1 (Accession #BAD67157); Cry30Ca2 (Accession #ACU24781); Cry30Da1 (Accession #EF095955); Cry30Db1 (Accession #BAE80088); Cry30Ea1 (Accession #ACC95445); Cry30Ea2 (Accession #FJ499389); Cry30Fa1 (Accession #ACI22625); Cry30Ga1 (Accession #ACG60020); Cry30Ga2 (Accession #HQ638217); Cry31Aa1 (Accession #BAB11757); Cry31Aa2 (Accession #AAL87458); Cry31Aa3 (Accession #BAE79808); Cry31Aa4 (Accession #BAF32571); Cry31Aa5 (Accession #BAF32572); Cry31Aa6 (Accession #BAI44026); Cry31Ab1 (Accession #BAE79809); Cry31Ab2 (Accession #BAF32570); Cry31Ac1 (Accession #BAF34368); Cry31Ac2 (Accession #AB731600); Cry31Ad1 (Accession #BAI44022); Cry32Aa1 (Accession #AAG36711); Cry32Aa2 (Accession #GU063849); Cry32Ab1 (Accession #GU063850); Cry32Ba1 (Accession #BAB78601); Cry32Ca1 (Accession #BAB78602); Cry32Cb1 (Accession #KC156708); Cry32Da1 (Accession #BAB78603); Cry32Ea1 (Accession #GU324274); Cry32Ea2 (Accession #KC156686); Cry32Eb1 (Accession #KC156663); Cry32Fa1 (Accession #KC156656); Cry32Ga1 (Accession #KC156657); Cry32Ha1 (Accession #KC156661); Cry32Hb1 (Accession #KC156666); Cry321a1 (Accession #KC156667); Cry32Ja1 (Accession #KC156685); Cry32Ka1 (Accession #KC156688); Cry32La1 (Accession #KC156689); Cry32Ma1 (Accession #KC156690); Cry32Mb1 (Accession #KC156704); Cry32Na1 (Accession #KC156691); Cry32Oa1 (Accession #KC156703); Cry32Pa1 (Accession #KC156705); Cry32Qa1 (Accession #KC156706); Cry32Ra1 (Accession #KC156707); Cry32Sa1 (Accession #KC156709); Cry32Ta1 (Accession #KC156710); Cry32Ua1 (Accession #KC156655); Cry33Aa1 (Accession #AAL26871); Cry34Aa1 (Accession #AAG50341); Cry34Aa2 (Accession #AAK64560); Cry34Aa3 (Accession #AAT29032); Cry34Aa4 (Accession #AAT29030); Cry34Ab1 (Accession #AAG41671); Cry34Ac1 (Accession #AAG50118); Cry34Ac2 (Accession #AAK64562); Cry34Ac3 (Accession #AAT29029); Cry34Ba1 (Accession #AAK64565); Cry34Ba2 (Accession #AAT29033); Cry34Ba3 (Accession #AAT29031); Cry35Aa1 (Accession #AAG50342); Cry35Aa2 (Accession #AAK64561); Cry35Aa3 (Accession #AAT29028); Cry35Aa4 (Accession #AAT29025); Cry35Ab1 (Accession #AAG41672); Cry35Ab2 (Accession #AAK64563); Cry35Ab3 (Accession #AY536891); Cry35Ac1 (Accession #AAG50117); Cry35Ba1 (Accession #AAK64566); Cry35Ba2 (Accession #AAT29027); Cry35Ba3 (Accession #AAT29026); Cry36Aa1 (Accession #AAK64558); Cry37Aa1 (Accession #AAF76376); Cry38Aa1 (Accession #AAK64559); Cry39Aa1 (Accession #BAB72016); Cry40Aa1 (Accession #BAB72018); Cry40Ba1 (Accession #BAC77648); Cry40Ca1 (Accession #EU381045); Cry40Da1 (Accession #ACF15199); Cry41Aa1 (Accession #BAD35157); Cry41Ab1 (Accession #BAD35163); Cry41Ba1 (Accession #HM461871); Cry41Ba2 (Accession #ZP_04099652); Cry42Aa1 (Accession #BAD35166); Cry43Aa1 (Accession #BAD15301); Cry43Aa2 (Accession #BAD95474); Cry43Ba1 (Accession #BAD15303); Cry43Ca1 (Accession #KC156676); Cry43Cb1 (Accession #KC156695); Cry43Cc1 (Accession #KC156696); Cry43-like (Accession #BAD15305); Cry44Aa (Accession #BAD08532); Cry45Aa (Accession #BAD22577); Cry46Aa (Accession #BAC79010); Cry46Aa2 (Accession #BAG68906); Cry46Ab (Accession #BAD35170); Cry47Aa (Accession #AAY24695); Cry48Aa (Accession #CAJ18351); Cry48Aa2 (Accession #CAJ86545); Cry48Aa3 (Accession #CAJ86546); Cry48Ab (Accession #CAJ86548); Cry48Ab2 (Accession #CAJ86549); Cry49Aa (Accession #CAH56541); Cry49Aa2 (Accession #CAJ86541); Cry49Aa3 (Accession #CAJ86543); Cry49Aa4 (Accession #CAJ86544); Cry49Ab1 (Accession #CAJ86542); Cry50Aa1 (Accession #BAE86999); Cry50Ba1 (Accession #GU446675); Cry50Ba2 (Accession #GU446676); Cry51Aa1 (Accession #ABI14444); Cry51Aa2 (Accession #GU570697); Cry52Aa1 (Accession #EF613489); Cry52Ba1 (Accession #FJ361760); Cry53Aa1 (Accession #EF633476); Cry53Ab1 (Accession #FJ361759); Cry54Aa1 (Accession #ACA52194); Cry54Aa2 (Accession #GQ140349); Cry54Ba1 (Accession #GU446677); Cry55Aa1 (Accession #ABW88932); Cry54Ab1 (Accession #JQ916908); Cry55Aa2 (Accession #AAE33526); Cry56Aa1 (Accession #ACU57499); Cry56Aa2 (Accession #GQ483512); Cry56Aa3 (Accession #JX025567); Cry57Aa1 (Accession #ANC87261); Cry58Aa1 (Accession #ANC87260); Cry59Ba1 (Accession #JN790647); Cry59Aa1 (Accession #ACR43758); Cry60Aa1 (Accession #ACU24782); Cry60Aa2 (Accession #EA057254); Cry60Aa3 (Accession #EEM99278); Cry60Ba1 (Accession #GU810818); Cry60Ba2 (Accession #EA057253); Cry60Ba3 (Accession #EEM99279); Cry61Aa1 (Accession #HM035087); Cry61Aa2 (Accession #HM132125); Cry61Aa3 (Accession #EEM19308); Cry62Aa1 (Accession #HM054509); Cry63Aa1 (Accession #BA144028); Cry64Aa1 (Accession #BAJ05397); Cry65Aa1 (Accession #HM461868); Cry65Aa2 (Accession #ZP_04123838); Cry66Aa1 (Accession #HM485581); Cry66Aa2 (Accession #ZP_04099945); Cry67Aa1 (Accession #HM485582); Cry67Aa2 (Accession #ZP_04148882); Cry68Aa1 (Accession #HQ113114); Cry69Aa1 (Accession #HQ401006); Cry69Aa2 (Accession #JQ821388); Cry69Ab1 (Accession #JN209957); Cry70Aa1 (Accession #JN646781); Cry70Ba1 (Accession #ADO51070); Cry70Bb1 (Accession #EEL67276); Cry71Aa1 (Accession #JX025568); Cry72Aa1 (Accession #JX025569); Cyt1Aa (GenBank Accession Number X03182); Cyt1Ab (GenBank Accession Number X98793); Cyt1B (GenBank Accession Number U37196); Cyt2A (GenBank Accession Number Z14147); and Cyt2B (GenBank Accession Number U52043).

Examples of β-endotoxins also include but are not limited to Cry1A proteins of U.S. Pat. Nos. 5,880,275 and 7,858,849; a DIG3 or DIG11 toxin (N-terminal deletion of α-helix 1 and/or α-helix 2 variants of cry proteins such as Cry1A, Cry3A) of U.S. Pat. Nos. 8,304,604, 8,304,605 and 8,476,226; Cry1B of U.S. patent application Ser. No. 10/525,318; Cry1C of U.S. Pat. No. 6,033,874; Cry1F of U.S. Pat. Nos. 5,188,960 and 6,218,188; Cry1A/F chimeras of U.S. Pat. Nos. 7,070,982; 6,962,705 and 6,713,063; a Cry2 protein such as Cry2Ab protein of U.S. Pat. No. 7,064,249); a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as members of the Cry9A, Cry9B, Cry9C, Cry9D, Cry9E and Cry9F families; a Cry15 protein of Naimov, et al., (2008) Applied and Environmental Microbiology, 74:7145-7151; a Cry22, a Cry34Ab1 protein of U.S. Pat. Nos. 6,127,180, 6,624,145 and 6,340,593; a CryET33 and cryET34 protein of U.S. Pat. Nos. 6,248,535, 6,326,351, 6,399,330, 6,949,626, 7,385,107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US Patent Application Publication Number 2008/0295207; ET29, ET37, TIC809, TIC810, TIC812, TIC127, TIC128 of PCT US 2006/033867; AXMI027, AXMI036, and AXMI038 of U.S. Pat. No. 8,236,757; AXMI031, AXMI039, AXMI040, AXMI049 of U.S. Pat. No. 7,923,602; AXMI018, AXMI020 and AXMI021 of WO 2006/083891; AXMI010 of WO 2005/038032; AXMI003 of WO 2005/021585; AXMI008 of US Patent Application Publication Number 2004/0250311; AXMI006 of US Patent Application Publication Number 2004/0216186; AXMI007 of US Patent Application Publication Number 2004/0210965; AXMI009 of US Patent Application Number 2004/0210964; AXMI014 of US Patent Application Publication Number 2004/0197917; AXMI004 of US Patent Application Publication Number 2004/0197916; AXMI028 and AXMI029 of WO 2006/119457; AXMI007, AXMI008, AXMI0080rf2, AXMI009, AXMI014 and AXMI004 of WO 2004/074462; AXMI150 of U.S. Pat. No. 8,084,416; AXMI205 of US Patent Application Publication Number 2011/0023184; AXMI011, AXMI012, AXMI013, AXMI015, AXMI019, AXMI044, AXMI037, AXMI043, AXMI033, AXMI034, AXMI022, AXMI023, AXMI041, AXMI063 and AXMI064 of US Patent Application Publication Number 2011/0263488; AXMI-R1 and related proteins of US Patent Application Publication Number 2010/0197592; AXM221Z, AXM222z, AXM223z, AXM224z and AXM225z of WO 2011/103248; AXM218, AXM219, AXM220, AXM226, AXM227, AXM228, AXM229, AXM230 and AXM231 of WO 2011/103247; AXMI115, AXMI113, AXMI005, AXMI163 and AXMI184 of U.S. Pat. No. 8,334,431; AXMI001, AXMI002, AXMI030, AXMI035 and AXMI045 of US Patent Application Publication Number 2010/0298211; AXMI066 and AXMI076 of US Patent Application Publication Number 2009/0144852; AXM128, AXM130, AXM131, AXM133, AXM140, AXM141, AXM142, AXM143, AXM144, AXM146, AXM148, AXM149, AXM152, AXM153, AXM154, AXM155, AXM156, AXM157, AXM158, AXM162, AXM165, AXM166, AXM167, AXM168, AXM169, AXM170, AXM171, AXM172, AXM173, AXM174, AXM175, AXM176, AXM177, AXM178, AXM179, AXM180, AXM181, AXM182, AXM185, AXM186, AXM187, AXM188, AXM189 of U.S. Pat. No. 8,318,900; AXM079, AXM080, AXM081, AXM082, AXM091, AXM092, AXM096, AXM097, AXM098, AXM099, AXM100, AXM101, AXM102, AXM103, AXM104, AXM107, AXM108, AXM109, AXM110, AXM111, AXM112, AXM114, AXM116, AXM117, AXM118, AXM119, AXM120, AXM121, AXM122, AXM123, AXM124, AXM1257, AXM1268, AXM127, AXM129, AXM164, AXM151, AXM161, AXM183, AXM132, AXM138, AXM137 of US Patent Application Publication Number 2010/0005543, AXM232, AXM233 and AXM249 of US Patent Application Publication Number 201400962281; cry proteins such as Cry1A and Cry3A having modified proteolytic sites of U.S. Pat. No. 8,319,019; a Cry1Ac, Cry2Aa and Cry1Ca toxin protein from Bacillus thuringiensis strain VBTS2528 of US Patent Application Publication Number 2011/0064710. Other Cry proteins are well known to one skilled in the art (see, Crickmore, et al., “Bacillus thuringiensis toxin nomenclature” (2011), at lifesci.sussex.ac.uk/home/Neil_Crickmore/Bt/ which can be accessed on the world-wide web using the “www” prefix). The insecticidal activity of Cry proteins is well known to one skilled in the art (for review, see, van Frannkenhuyzen, (2009) J. Invert. Path. 101:1-16). The use of Cry proteins as transgenic plant traits is well known to one skilled in the art and Cry-transgenic plants including but not limited to plants expressing Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, Cry9c and CBI-Bt have received regulatory approval (see, Sanahuja, (2011) Plant Biotech Journal 9:283-300 and the CERA. (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). More than one pesticidal proteins well known to one skilled in the art can also be expressed in plants such as Vip3Ab & Cry1Fa (US2012/0317682); Cry1BE & Cry1F (US2012/0311746); Cry1CA & Cry1AB (US2012/0311745); Cry1F & CryCa (US2012/0317681); Cry1DA & Cry1BE (US2012/0331590); Cry1DA & Cry1Fa (US2012/0331589); Cry1AB & Cry1BE (US2012/0324606); Cry1Fa & Cry2Aa and Cry1I & Cry1E (US2012/0324605); Cry34Ab/35Ab and Cry6Aa (US20130167269); Cry34Ab/VCry35Ab & Cry3Aa (US20130167268); and Cry3A and Cry1Ab or Vip3Aa (US20130116170). Pesticidal proteins also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Pesticidal proteins also include VIP (vegetative insecticidal proteins) toxins of U.S. Pat. Nos. 5,877,012, 6,107,279 6,137,033, 7,244,820, 7,615,686, and 8,237,020 and the like. Other VIP proteins are well known to one skilled in the art (see, lifesci.sussex.ac.uk/home/Neil_Crickmore/Bt/vip.html which can be accessed on the world-wide web using the “www” prefix). Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Pesticidal proteins also include spider, snake and scorpion venom proteins. Examples of spider venom peptides include but are not limited to lycotoxin-1 peptides and mutants thereof (U.S. Pat. No. 8,334,366).

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

### B. Recombinant DNA Constructs

Also provided are recombinant DNA constructs comprising any of the polynucleotides described herein. In certain embodiments, the recombinant DNA construct further comprises at least one regulatory element. In certain embodiments the at least one regulatory element is a heterologous regulatory element. In certain embodiments, the at least one regulatory element of the recombinant DNA construct comprises a promoter. In certain embodiments, the promoter is a heterologous promoter.

A number of promoters can be used in recombinant DNA constructs of the present disclosure. The promoters can be selected based on the desired outcome, and may include constitutive, tissue-specific, inducible, or other promoters for expression in the host organism.

A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

A tissue-specific or developmentally-regulated promoter is a DNA sequence which regulates the expression of a DNA sequence selectively in the cells/tissues of a plant, such as in those cells/tissues critical to tassel development, seed set, or both, and which usually limits the expression of such a DNA sequence to the developmental period of interest (e.g. tassel development or seed maturation) in the plant. Any identifiable promoter which causes the desired temporal and spatial expression may be used in the methods of the present disclosure.

Many leaf-preferred promoters are known in the art (Yamamoto et al. (1997) Plant J. 12(2):255-265; Kwon et al. (1994) Plant Physiol. 105:357-367; Yamamoto et al. (1994) Plant Cell Physiol. 35(5):773-778; Gotor et al. (1993) Plant J. 3:509-518; Orozco et al. (1993) Plant Mol. Biol. 23(6):1129-1138; and Matsuoka et al. (1993) Proc. Natl. Acad. Sci. USA 90(20):9586-9590).

Promoters which are seed or embryo-specific and may be useful in the disclosure include soybean Kunitz trypsin inhibitor (Kti3, Jofuku and Goldberg. (1989) Plant Cell 1:1079-1093), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564). Promoters of seed-specific genes operably linked to heterologous coding regions in chimeric gene constructions maintain their temporal and spatial expression pattern in transgenic plants. Such examples include Arabidopsis 2S seed storage protein gene promoter to express enkephalin peptides in Arabidopsis and Brassica napus seeds (Vanderkerckhove et al. (1989) Bio/Technology 7: L929-932), bean lectin and bean beta-phaseolin promoters to express luciferase (Riggs et al. (1989) Plant Sci. 63:47-57), and wheat glutenin promoters to express chloramphenicol acetyl transferase (Colot et al. (1987) EMBO J 6:3559-3564).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Also contemplated are synthetic promoters which include a combination of one or more heterologous regulatory elements.

The promoter of the recombinant DNA constructs of the invention can be any type or class of promoter known in the art, such that any one of a number of promoters can be used to express the various polynucleotide sequences disclosed herein, including the native promoter of the polynucleotide sequence of interest. The promoters for use in the recombinant DNA constructs of the invention can be selected based on the desired outcome.

The recombinant DNA constructs of the present disclosure may also include other regulatory elements, including but not limited to, translation leader sequences, introns, and polyadenylation recognition sequences. In certain embodiments, a recombinant DNA construct further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg. (1988) Mol. Cell Biol. 8:4395-4405; Callis et al. (1987) Genes Dev. 1:1183-1200).

### C. Plants and Plant Cells

Provided are plants, plant cells, plant parts, seed and grain comprising in its genome any of the recombinant DNA constructs described herein, so that the plants, plant cells, plant parts, seed, and/or grain have increased expression of the encoded polypeptide.

Also provided are plants, plant cells, plant parts, seeds, and grain comprising an introduced genetic modification at a genomic locus that encodes a polypeptide comprising an amino acid sequence that is at least 80% identical to an amino acid sequence selected from the group consisting of SEQ ID NOs: 3, 6, 9, 12, 15, 18, 21 or 24. In certain embodiments, the genetic modification increases the activity of the encoded polypeptide. In certain embodiments, the genetic modification increases the level of the encoded polypeptide. In certain embodiments, the genetic modification increases both the level and activity of the encoded polypeptide.

The plant may be a monocotyledonous or dicotyledonous plant, for example, a rice or maize or soybean plant, such as a maize hybrid plant or a maize inbred plant. The plant may also be sunflower, sorghum, canola, wheat, alfalfa, cotton, barley, millet, sugar cane or switchgrass.

In certain embodiments the plant exhibits increased insect tolerance when compared to a control plant.

D. Stacking with Other Traits of Interest

In some embodiments, the inventive polynucleotides disclosed herein are engineered into a molecular stack. Thus, the various host cells, plants, plant cells, plant parts, seeds, and/or grain disclosed herein can further comprise one or more traits of interest. In certain embodiments, the host cell, plant, plant part, plant cell, seed, and/or grain is stacked with any combination of polynucleotide sequences of interest in order to create plants with a desired combination of traits. As used herein, the term “stacked” refers to having multiple traits present in the same plant or organism of interest. For example, “stacked traits” may comprise a molecular stack where the sequences are physically adjacent to each other. A trait, as used herein, refers to the phenotype derived from a particular sequence or groups of sequences. In one embodiment, the molecular stack comprises at least one polynucleotide that confers tolerance to glyphosate. Polynucleotides that confer glyphosate tolerance are known in the art.

In certain embodiments, the molecular stack comprises at least one polynucleotide that confers tolerance to glyphosate and at least one additional polynucleotide that confers tolerance to a second herbicide.

The plant, plant cell, plant part, seed, and/or grain having an inventive polynucleotide sequence can also be combined with at least one other trait to produce plants that further comprise a variety of desired trait combinations. For instance, the plant, plant cell, plant part, seed, and/or grain having an inventive polynucleotide sequence may be stacked with polynucleotides encoding polypeptides having pesticidal and/or insecticidal activity, or a plant, plant cell, plant part, seed, and/or grain having an inventive polynucleotide sequence may be combined with a plant disease resistance gene.

Transgenic plants may comprise a stack of one or more insecticidal or insect tolerance polynucleotides disclosed herein with one or more additional polynucleotides resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene and cotransformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Co-transformation of genes can be carried out using single transformation vectors comprising multiple genes or genes carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853, all of which are herein incorporated by reference.

### Methods:

Provided is a method for increasing insect tolerance in a plant, comprising increasing the expression of at least one polynucleotide encoding a polypeptide with amino acid sequence of at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 3, 6, 9, 12, 15, 18, 21 or 24.

In certain embodiments, the method comprises: (a) expressing in a regenerable plant cell a recombinant DNA construct comprising a regulatory element operably linked to the polynucleotide encoding the polypeptide; and (b) generating the plant, wherein the plant comprises in its genome the recombinant DNA construct. In certain embodiments the regulatory element is a heterologous promoter.

In certain embodiments, the method comprises: (a) introducing in a regenerable plant cell a targeted genetic modification at a genomic locus that encodes the polypeptide; and (b) generating the plant, wherein the level and/or activity of the encoded polypeptide is increased in the plant. In certain embodiments the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), engineered site-specific meganucleases, or Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at least 80% identical to an amino acid sequence selected from the group consisting of SEQ ID NOs: 3, 6, 9, 12, 15, 18, 21 or 24.

In certain embodiments the DNA modification is an insertion of one or more nucleotides, preferably contiguous, in the genomic locus. For example, the insertion of an expression modulating element (EME), such as an EME described in PCT/US2018/025446, in operable linkage with the gene. In certain embodiments, the targeted DNA modification may be the replacement of the endogenous polypeptide promoter with another promoter known in the art to have higher expression. In certain embodiments, the targeted DNA modification may be the insertion of a promoter known in the art to have higher expression into the 5′UTR so that expression of the endogenous polypeptide is controlled by the inserted promoter. In certain embodiments, the DNA modification is a modification to optimize Kozak context to increase expression. In certain embodiments, the DNA modification is a polynucleotide modification or SNP at a site that regulates the stability of the expressed protein.

The plant for use in the inventive methods can be any plant species described herein. In certain embodiments, the plant is maize, soybean, or rice.

Various methods can be used to introduce a sequence of interest into a plant, plant part, plant cell, seed, and/or grain. “Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In other embodiments, the inventive polynucleotides disclosed herein may be introduced into plants by contacting plants with a virus or viral nucleic acids. Generally, such methods involve incorporating a nucleotide construct of the disclosure within a DNA or RNA molecule. It is recognized that the inventive polynucleotide sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide disclosed herein, for example, as part of an expression cassette, stably incorporated into their genome.

Transformed plant cells which are derived by plant transformation techniques, including those discussed above, can be cultured to regenerate a whole plant which possesses the transformed genotype (i.e., an inventive polynucleotide), and thus the desired phenotype, such as increased yield. For transformation and regeneration of maize see, Gordon-Kamm et al., The Plant Cell, 2:603-618 (1990).

Various methods can be used to introduce a genetic modification at a genomic locus that encodes a polypeptide disclosed herein into the plant, plant part, plant cell, seed, and/or grain. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In some embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

In addition to modification by a double strand break technology, modification of one or more bases without such double strand break are achieved using base editing technology, see e.g., Gaudelli et al., (2017) Programmable base editing of A*T to G*C in genomic DNA without DNA cleavage. Nature 551(7681):464-471; Komor et al., (2016) Programmable editing of a target base in genomic DNA without double-stranded DNA cleavage, Nature 533(7603):420-4.

These fusions contain dCas9 or Cas9 nickase and a suitable deaminase, and they can convert e.g., cytosine to uracil without inducing double-strand break of the target DNA.

Uracil is then converted to thymine through DNA replication or repair. Improved base editors that have targeting flexibility and specificity are used to edit endogenous locus to create target variations and improve grain yield. Similarly, adenine base editors enable adenine to inosine change, which is then converted to guanine through repair or replication. Thus, targeted base changes i.e., C•G to T•A conversion and A•T to G•C conversion at one more location made using appropriate site-specific base editors.

In an embodiment, base editing is a genome editing method that enables direct conversion of one base pair to another at a target genomic locus without requiring double-stranded DNA breaks (DSBs), homology-directed repair (HDR) processes, or external donor DNA templates. In an embodiment, base editors include (i) a catalytically impaired CRISPR-Cas9 mutant that are mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; and (iv) nickase activity to cleave the non-edited DNA strand, followed by cellular DNA repair processes to replace the G-containing DNA strand.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site.

The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

### EXAMPLES

The following are examples of specific embodiments of some aspects of the invention. The examples are offered for illustrative purposes only and are not intended to limit the scope of the invention in any way.

### Example 1

### Cloning and Vector Construction of Insect Tolerance Genes

A binary construct that contains four multimerized enhancers elements derived from the Cauliflower Mosaic Virus 35S (CaMV 35S) promoter was used, and the rice activation tagging population was developed from four japonica (Oryza sativa ssp. japonica) varieties (Zhonghua 11, Chaoyou 1, Taizhong 65 and Nipponbare), which were transformed by Agrobacteria-mediated transformation method as described by Lin and Zhang ((2005) Plant Cell Rep. 23:540-547). The transgenic lines generated were developed and the transgenic seeds were harvested to form the rice activation tagging population.

Insect tolerance tagging lines (ATLs) were confirmed in repeated field experiments and their T-DNA insertion loci were determined. The genes near by the left border and right border of the T-DNA were cloned and the functional genes were recapitulated by lab screens. Only the recapitulated functional genes are showed herein. And based on LOC IDs of these genes shown in Table 2, primers were designed for cloning the rice insect tolerance genes OsAAK1, OsDN-ITP8, OsPMR5, OsERV-B, OsbHLH065, OsGRP1, OsAP2-4, OsDUF630/DUF632.

TABLE 2
Rice gene names, Gene IDs (from TIGR) and Construct IDs

| Gene name | LOC ID | Construct ID |
|---|---|---|
| OsAAK1 | LOC_Os04g46460.2 | DP1931 |
| OsDN-ITP8 | LOC_Os03g16320.1 | DP2605 |
| OsPMR5 | LOC_Os12g01560.1 | DP1529 |
| OsERV-B | LOC_Os06g38450.1 | DP1552 |
| OsbHLH065 | LOC_Os04g41570.2 | DP1783 |
| OsGRP1 | LOC_Os04g41580.1 | DP1784 |
| OsAP2-4 | LOC_Os04g46440.1 | DP1948 |
| OsDUF630/DUF632 | LOC_Os02g07850.1 | DP2583 |

PCR amplified products were extracted after the agarose gel electrophoresis using a column kit and then ligated with TA cloning vectors. The sequences and orientation in these constructs were confirmed by sequencing. Each gene was cloned into a plant binary construct.

### Example 2

### Transformation of Transgenic Rice Lines

Zhonghua 11 (Oryza sativa L.) were transformed with either a vector prepared in Example 1 or an empty vector (DP0158) by Agrobacteria-mediated transformation as described by Lin and Zhang ((2005) Plant Cell Rep. 23:540-547). Transgenic seedlings (T₀) generated in the transformation laboratory were transplanted in field to get T₁ seeds. The T₁ and subsequent T₂ seeds were screened to confirm transformation and positively identified transgenic seeds were used in the following trait screens.

### Example 3

### Characterization of the Transgenic Rice Plants by ACB Assay

Asian corn borer (ACB) (Ostrinia furnacalis (Gueńe)) is an important insect pest of maize in Asia. This insect is distributed from China to Australia and the Solomon Islands. In northern parts of its range, the moths have one or a few generations per year, but in the tropics, generations are continuous and overlapping. The caterpillars can cause severe yield losses in corn, both by damage to the kernels and by feeding on the tassels, leaves, and stalks. Survival and growth of the caterpillar is highest on the reproductive parts of the plant. Other economic plants attacked include bell pepper, ginger and sorghum. Recently, the Asian corn borer appears to have become an important pest of cotton. A number of wild grasses are also used as hosts (D. M. Nafusa & I. H. Schreinera. 2012. Review of the biology and control of the Asian corn borer, Ostrinia furnacalis (Lep: Pyralidae). Tropical Pest Management. 37: 41-56).

ACB insect was used to identify rice plants which can inhibit larva development. Asian corn borer populations were obtained from the Institute of Plant Protection of Chinese Academy of Agricultural Sciences. This population was reared for more than 10 generations at 25-27° C., 60-80% relative humidity, under photo-period of 16L:8D. The larvae were fed with artificial diet (Zhou Darong, Ye Zhihua, Wang Zhenying, 1995), and the eggs were hatched in incubator at 27° C. The newly hatched larvae were used in assays.

T2 plants generated with the construct were tested in the assays for about three times with four to six repeats. The seedlings of ZH11-TC and DP0158 were used as controls. About ten lines transgenic rice and 450 seeds of each line were tested. All seeds were sterilized by 800 ppm carbendazol for 8 h at 32° C. and washed 3-5 times, then placed on a layer of wet gauze in petri dish (12×12 cm). The germinated seeds were cultured in distilled water at 28° C. for 10 days and the seedlings which were 8-10 cm in height were used to feed ACB larvae.

Randomized block design was used, and ten transgenic lines from a construct were tested in one experimental unit to evaluate the gene function by SAS PROC GLIMMIX considering construct, line and environment effects. If the larva growth inhibitory rates of the transgenic rice plants at both construct and line levels were significantly greater than controls (P<0.05), the gene was considered having ACB tolerant function.

The three largest larvae in each well were selected, compared with the larvae in the well with ZH11-TC seedlings, and then a tolerant value was obtained according to Table 3. If the larva in the control well developed to third instar, then the larval development was considered as normal and the tolerant value is 0; if the larva developed to second instar, it was smaller compared to the normal developed larva and the tolerant value is 1; and if the larva developed to first instar, it is very smaller and the tolerant value is 2.

TABLE 3
Scoring Scales for Asian corn borer

| Tolerant value | Instars of larva | Size of larva |
|---|---|---|
| 0 | 3^{rd} instar | Normal |
| 1 | 2^{nd} instar | Smaller |
| 2 | 1^{st} instar | Severe smaller |

Larva growth inhibitory rate was used as a parameter for ACB insect tolerance assay, which is the percentage of the inhibited larva number over the statistics number of larva, wherein the inhibited larva number is the sum of the tolerance value of test insects from wells and the statistics number of larva is the sum of the number of all the observed insects and number of larva at 1^{st} instar. Then the raw data was analyzed by Chi-square, the lines with P<0.01 were considered as ACB tolerance positive lines.

In order to investigate whether OsAAK1, OsDN-ITP8, OsPMR5, OsERV-B, OsbHLH065, OsGRP1, OsAP2-4, OsDUF630/DUF632 transgenic rice plants from Example 2 have insect tolerance trait, all the transgenic rice plants and ZH11-TC and DP0158 rice plants were tested against ACB insect.

### (1) ACB Screening Results of OsAAK1 Transgenic Rice Plants

OsAAK1 transgenic rice plants were tested three times. All the experiments showed that the average larva growth inhibitory rate of OsAAK1 transgenic rice plants was significantly greater than that of DP0158 control.

In the first experiment, ten OsAAK1 transgenic lines were placed on one plate, and repeated for 5 times. After ACB neonate larvae inoculating seedlings for 5 days, the seedlings of ZH11-TC and DP0158 were significantly damaged by ACB insects, while the OsAAK1 transgenic seedlings were less damaged, and the insects fed with the OsAAK1 transgenic seedlings was smaller than that fed with ZH11-TC and DP0158 controls. Five days after inoculation, 464 larvae were found, 16 larvae developed to 1^{st} instar, and 193 larvae developed to 2^{nd} instar. Two larvae in ZH11-TC seedlings' wells developed to 1^{st} instar, and 63 larvae developed to 2^{nd} instar; 5 larvae developed to 1^{St} instar and 55 larvae developed to 2^{nd} instar in DP0158 seedlings' wells. The average larva growth inhibitory rates of OsAAK1 transgenic rice, ZH11-TC and DP0158 were 47%, 40% and 37%, respectively. The average larva growth inhibitory rate of OsAAK1 transgenic rice was greater than that of ZH11-TC control and significantly greater than that of DP0158 control. These results show that over-expression of OsAAK1 in rice significantly increased ACB insect tolerance of transgenic rice at construct level.

Further analysis at transgenic line level is displayed in Table 4. Three transgenic lines exhibited significantly greater larva growth inhibitory rates than that of DP0158 control. These results further indicate OsAAK1 plays a role in increasing ACB insect tolerance in rice compared to controls at line level.

TABLE 4
ACB assay of OsAAK1 transgenic rice under laboratory screening condition

| | Number | Number | Number of | Larvae | | | | |
|---|---|---|---|---|---|---|---|---|
| | of larva | of larvae | total | growth | CK = ZH11-TC | | CK = DP0158 | |
| | at 1^{st} | at 2^{nd} | observed | inhibitory | P | | P | |
| Line ID | instar | instar | larvae | rate (%) | value | P ≤ 0.05 | value | P ≤ 0.05 |
| DP1931 | 16 | 193 | 464 | 46.88 | 0.1393 | | 0.0213 | Y |
| (Construct) | | | | | | | | |
| ZH11-TC | 2 | 63 | 164 | 40.36 | | | | |
| DP0158 | 5 | 55 | 172 | 36.72 | | | | |
| DP1931.01 | 1 | 22 | 44 | 53.33 | 0.1262 | | 0.0486 | Y |
| DP1931.03 | 1 | 18 | 39 | 50.00 | 0.2736 | | 0.1277 | |
| DP1931.04 | 3 | 21 | 49 | 51.92 | 0.1484 | | 0.0555 | |
| DP1931.06 | 2 | 21 | 44 | 54.35 | 0.0973 | | 0.0356 | Y |
| DP1931.10 | 1 | 14 | 49 | 32.00 | 0.2920 | | 0.5407 | |
| DP1931.11 | 2 | 17 | 48 | 42.00 | 0.8369 | | 0.4999 | |
| DP1931.12 | 1 | 23 | 51 | 48.08 | 0.3302 | | 0.1472 | |
| DP1931.13 | 2 | 20 | 41 | 55.81 | 0.0755 | | 0.0274 | Y |
| DP1931.14 | 1 | 18 | 45 | 43.48 | 0.7051 | | 0.4047 | |
| DP1931.15 | 2 | 19 | 54 | 41.07 | 0.9258 | | 0.5609 | |

### (2) ACB Screening Results of OsDN-ITP8 Transgenic Rice Plants

OsDN-ITP8 transgenic rice plants were tested three times. All the experiments showed that the average larva growth inhibitory rate of OsDN-ITP8 transgenic rice plants was greater than that of ZH11-TC and DP0158 controls. And two of them significantly greater than that of ZH11-TC control, and one of them significantly greater than that of DP0158 control.

In the third experiment, ten OsDN-ITP8 transgenic lines were placed on one 32-well plate with 6 repeats. Five days after inoculation, 624 larvae were found, 6 larvae developed to 1^{st} instar, and 316 larvae developed to 2^{nd} instar. While 60 of 203 larvae in ZH11-TC seedlings' wells developed to 2^{nd} instar; 1 of 204 larvae developed to 1^{st} instar and 71 larvae developed to 2^{nd} instar in DP0158 seedlings' wells. The average larva growth inhibitory rates of OsDN-ITP8 transgenic rice, ZH11-TC and DP0158 were 52%, 30% and 36%, respectively. The average larva growth inhibitory rate of OsDN-ITP8 transgenic rice was significantly greater than that of ZH11-TC and DP0158 controls. These results show that over-expression of OsDN-ITP8 in rice significantly increased ACB insect tolerance of transgenic rice at construct level.

Further analysis at transgenic line level is displayed in Table 5. Ten transgenic lines exhibited significantly greater larva growth inhibitory rates than that of ZH11-TC control; and seven lines exhibited significantly greater larva growth inhibitory rates than that of DP0158 control. These results further indicate OsDN-ITP8 plays a role in increasing ACB insect tolerance in rice compared to controls at line level.

TABLE 5
ACB assay of OsDN-ITP8 transgenic rice under laboratory screening condition

| | Number | Number | Number of | Larvae | | | | |
|---|---|---|---|---|---|---|---|---|
| | of larva | of larvae | total | growth | CK = ZH11-TC | | CK = DP0158 | |
| | at 1^{st} | at 2^{nd} | observed | inhibitory | P | | P | |
| Line ID | instar | instar | larvae | rate (%) | value | P ≤ 0.05 | value | P ≤ 0.05 |
| DP2605 | 6 | 316 | 624 | 52.06 | 0.0000 | Y | 0.001 | Y |
| (Construct) | | | | | | | | |
| ZH11-TC | 0 | 60 | 203 | 29.56 | | | | |
| DP0158 | 1 | 71 | 204 | 35.61 | | | | |
| DP2605.01 | 0 | 37 | 60 | 61.67 | 0.0000 | Y | 0.0006 | Y |
| DP2605.02 | 0 | 31 | 63 | 49.21 | 0.0057 | Y | 0.0569 | Y |
| DP2605.03 | 0 | 34 | 66 | 51.52 | 0.0019 | Y | 0.0248 | Y |
| DP2605.04 | 1 | 33 | 67 | 51.47 | 0.0018 | Y | 0.0247 | Y |
| DP2605.05 | 1 | 29 | 59 | 51.67 | 0.0029 | Y | 0.0319 | Y |
| DP2605.06 | 0 | 28 | 63 | 44.44 | 0.0340 | Y | 0.2191 | |
| DP2605.07 | 0 | 30 | 63 | 47.62 | 0.0099 | Y | 0.0877 | |
| DP2605.08 | 0 | 37 | 59 | 62.71 | 0.0000 | Y | 0.0005 | Y |
| DP2605.09 | 2 | 31 | 66 | 51.47 | 0.0016 | Y | 0.0223 | Y |
| DP2605.10 | 2 | 26 | 58 | 50.00 | 0.0057 | Y | 0.0557 | |

### (3) ACB Screening Results of OsPMR5 Transgenic Rice Plants

OsPMR5 transgenic rice plants were tested three times. All the experiments showed that the average larva growth inhibitory rate of OsPMR5 transgenic rice plants was greater than that of DP0158 controls and significantly greater than that of ZH11-TC control.

In the first experiment, ten OsPMR5 transgenic lines were placed on one 32-well plate with 5 repeats. Five days after inoculation, 373 larvae were found, 13 larvae developed to 1^{st} instar, and 140 larvae developed to 2^{nd} instar. While 2 of 140 larvae in ZH11-TC seedlings' wells developed to 1^{St} instar and 31 larvae developed to 2^{nd} instar; 3 of 148 larvae developed to 1^{St} instar and 52 larvae developed to 2^{nd} instar in DP0158 seedlings' wells. The average larva growth inhibitory rates of OsPMR5 transgenic rice, ZH11-TC and DP0158 were 43%, 25% and 38%, respectively. The average larva growth inhibitory rate of OsPMR5 transgenic rice was significantly greater than that of ZH11-TC and DP0158 controls. These results show that over-expression of OsPMR5 in rice significantly increased ACB insect tolerance of transgenic rice at construct level.

Further analysis at transgenic line level is displayed in Table 6. Six transgenic lines exhibited significantly greater larva growth inhibitory rates than that of ZH11-TC control. These results further indicate OsPMR5 plays a role in increasing ACB insect tolerance in rice compared to controls at line level.

TABLE 6
ACB assay of OsPMR5 transgenic rice under laboratory screening condition

| | Number | Number | Number of | Larvae | | | | |
|---|---|---|---|---|---|---|---|---|
| | of larva | of larvae | total | growth | CK = ZH11-TC | | CK = DP0158 | |
| | at 1^{st} | at 2^{nd} | observed | inhibitory | P | | P | |
| Line ID | instar | instar | larvae | rate (%) | value | P ≤ 0.05 | value | P ≤ 0.05 |
| DP1529 | 13 | 140 | 373 | 43.01 | 0.0004 | Y | 0.3667 | |
| (Construct) | | | | | | | | |
| ZH11-TC | 2 | 31 | 140 | 24.65 | | | | |
| DP0158 | 3 | 52 | 148 | 38.41 | | | | |
| DP1529.06 | 1 | 17 | 38 | 48.72 | 0.0061 | Y | 0.2489 | |
| DP1529.07 | 1 | 14 | 31 | 50.00 | 0.0075 | Y | 0.2322 | |
| DP1529.08 | 0 | 13 | 34 | 38.24 | 0.1188 | | 0.9850 | |
| DP1529.09 | 2 | 18 | 41 | 51.16 | 0.0021 | Y | 0.1414 | |
| DP1529.12 | 2 | 17 | 37 | 53.85 | 0.0013 | Y | 0.0890 | |
| DP1529.13 | 3 | 6 | 32 | 34.29 | 0.2546 | | 0.6519 | |
| DP1529.14 | 1 | 10 | 36 | 32.43 | 0.3434 | | 0.5036 | |
| DP1529.17 | 2 | 11 | 45 | 31.91 | 0.3329 | | 0.4243 | |
| DP1529.20 | 1 | 12 | 35 | 38.89 | 0.0960 | Y | 0.9579 | |
| DP1529.21 | 0 | 22 | 44 | 50.00 | 0.0029 | Y | 0.1761 | |

### (4) ACB Screening Results of OsERV-B Transgenic Rice Plants

OsERV-B transgenic rice plants were tested three times. Two experiments showed that the average larva growth inhibitory rate of OsERV-B transgenic rice plants was greater than that of ZH11-TC control and significantly greater than that of DP0158 controls.

In the third experiment, ten OsERV-B transgenic lines were placed on one 32-well plate with 6 repeats. Five days after inoculation, 554 larvae were found, 71 larvae developed to 1^{st} instar, and 278 larvae developed to 2^{nd} instar. While 4 of 194 larvae in ZH11-TC seedlings' wells developed to 1^{st} instar and 108 larvae developed to 2^{nd} instar; 10 of 192 larvae developed to 1^{st} instar and 98 larvae developed to 2^{nd} instar in DP0158 seedlings' wells. The average larva growth inhibitory rates of OsERV-B transgenic rice, ZH11-TC and DP0158 were 67%, 59% and 58%, respectively. The average larva growth inhibitory rate of OsERV-B transgenic rice was significantly greater than that of ZH11-TC and DP0158 controls. These results show that over-expression of OsERV-B in rice significantly increased ACB insect tolerance of transgenic rice at construct level.

Further analysis at transgenic line level is displayed in Table 7. Three transgenic lines exhibited significantly greater larva growth inhibitory rates than that of ZH11-TC and DP0158 controls. These results further indicate OsERV-B plays a role in increasing ACB insect tolerance in rice compared to controls at line level.

TABLE 7
ACB assay of OsERV-B transgenic rice under laboratory screening condition

| | Number | Number | Number of | Larvae | | | | |
|---|---|---|---|---|---|---|---|---|
| | of larva | of larvae | total | growth | CK = ZH11-TC | | CK = DP0158 | |
| | at 1^{st} | at 2^{nd} | observed | inhibitory | P | | P | |
| Line ID | instar | instar | larvae | rate (%) | value | P ≤ 0.05 | value | P ≤ 0.05 |
| DP1552 | 71 | 278 | 554 | 67.20 | 0.0170 | Y | 0.0157 | Y |
| (Construct) | | | | | | | | |
| ZH11-TC | 4 | 108 | 194 | 58.59 | | | | |
| DP0158 | 10 | 98 | 192 | 58.42 | | | | |
| DP1552.01 | 6 | 23 | 46 | 67.31 | 0.1194 | | 0.1173 | |
| DP1552.02 | 5 | 32 | 57 | 67.74 | 0.1609 | | 0.1580 | |
| DP1552.03 | 10 | 27 | 54 | 73.44 | 0.0693 | | 0.0678 | |
| DP1552.04 | 10 | 30 | 55 | 76.92 | 0.0087 | Y | 0.0084 | Y |
| DP1552.06 | 11 | 22 | 58 | 63.77 | 0.4855 | | 0.4798 | |
| DP1552.10 | 14 | 31 | 54 | 86.76 | 0.0001 | Y | 0.0001 | Y |
| DP1552.11 | 3 | 31 | 65 | 54.41 | 0.5197 | | 0.5233 | |
| DP1552.13 | 3 | 24 | 51 | 55.56 | 0.7011 | | 0.7053 | |
| DP1552.15 | 1 | 25 | 54 | 49.09 | 0.1499 | | 0.1508 | |
| DP1552.18 | 8 | 33 | 60 | 72.06 | 0.0341 | Y | 0.0330 | Y |

### (5) ACB Screening Results of OsbHLH065 Transgenic Rice Plants

OsbHLH065 transgenic rice plants were tested three times. Two experiments showed that the average larva growth inhibitory rate of OsbHLH065 transgenic rice plants was significantly greater than that of ZH11-TC and DP0158 controls.

In the third experiment, ten OsbHLH065 transgenic lines were placed on one 32-well plate with 6 repeats. Five days after inoculation, 579 larvae were found, 9 larvae developed to 1^{st} instar, and 238 larvae developed to 2^{nd} instar. While 3 of 189 larvae in ZH11-TC seedlings' wells developed to 1^{st} instar and 45 larvae developed to 2^{nd} instar; 42 of 195 larvae developed to 2^{nd} instar in DP0158 seedlings' wells. The average larva growth inhibitory rates of OsbHLH065 transgenic rice, ZH11-TC and DP0158 were 44%, 27% and 22%, respectively. The average larva growth inhibitory rate of OsbHLH065 transgenic rice was significantly greater than that of ZH11-TC and DP0158 controls. These results show that over-expression of OsbHLH065 in rice significantly increased ACB insect tolerance of transgenic rice at construct level.

Further analysis at transgenic line level is displayed in Table 8. Eight transgenic lines exhibited significantly greater larva growth inhibitory rates than that of ZH11-TC and DP0158 controls. These results further indicate OsbHLH065 plays a role in increasing ACB insect tolerance in rice compared to controls at line level.

TABLE 8
ACB assay of OsbHLH065 transgenic rice under laboratory screening condition

| | Number | Number | Number of | Larvae | | | | |
|---|---|---|---|---|---|---|---|---|
| | of larva | of larvae | total | growth | CK = ZH11-TC | | CK = DP0158 | |
| | at 1^{st} | at 2^{nd} | observed | inhibitory | P | | P | |
| Line ID | instar | instar | larvae | rate (%) | value | P ≤ 0.05 | value | P ≤ 0.05 |
| DP1783 | 9 | 238 | 579 | 43.54 | 0.0001 | Y | 0.0001 | Y |
| (Construct) | | | | | | | | |
| ZH11-TC | 3 | 45 | 189 | 26.56 | | | | |
| DP0158 | 0 | 42 | 195 | 21.54 | | | | |
| DP1783.01 | 1 | 18 | 57 | 34.48 | 0.2479 | | 0.0501 | |
| DP1783.02 | 2 | 21 | 55 | 43.86 | 0.0155 | Y | 0.0015 | Y |
| DP1783.08 | 0 | 29 | 53 | 54.72 | 0.0003 | Y | 0.0001 | Y |
| DP1783.09 | 1 | 34 | 63 | 56.25 | 0.0001 | Y | 0.0001 | Y |
| DP1783.11 | 0 | 23 | 57 | 40.35 | 0.0497 | Y | 0.0061 | Y |
| DP1783.13 | 1 | 26 | 64 | 43.08 | 0.0164 | Y | 0.0014 | Y |
| DP1783.14 | 4 | 23 | 60 | 48.44 | 0.0020 | Y | 0.0001 | Y |
| DP1783.15 | 0 | 25 | 57 | 43.86 | 0.0159 | Y | 0.0015 | Y |
| DP1783.17 | 0 | 15 | 54 | 27.78 | 0.8450 | | 0.3297 | |
| DP1783.19 | 0 | 24 | 59 | 40.68 | 0.0419 | Y | 0.0048 | Y |

### (6) ACB Screening Results of OsGRP1 Transgenic Rice Plants

OsGRP1 transgenic rice plants were tested three times. Two experiments showed that the average larva growth inhibitory rate of OsGRP1 transgenic rice plants was significantly greater than that of ZH11-TC and DP0158 controls.

In the first experiment, ten OsGRP1 transgenic lines were placed on one 32-well plate with 5 repeats. Five days after inoculation, 332 larvae were found, 48 larvae developed to 1^{st} instar, and 139 larvae developed to 2^{nd} instar. While 10 of 123 larvae in ZH11-TC seedlings' wells developed to 1^{St} instar and 43 larvae developed to 2^{nd} instar; 11 of 120 larvae developed to 1^{St} instar and 30 larvae developed to 2^{nd} instar in DP0158 seedlings' wells. The average larva growth inhibitory rates of OsGRP1 transgenic rice, ZH11-TC and DP0158 were 62%, 47% and 40%, respectively. The average larva growth inhibitory rate of OsGRP1 transgenic rice was significantly greater than that of ZH11-TC and DP0158 controls. These results show that over-expression of OsGRP1 in rice significantly increased ACB insect tolerance of transgenic rice at construct level.

Further analysis at transgenic line level is displayed in Table 9. Four transgenic lines exhibited significantly greater larva growth inhibitory rates than that of ZH11-TC control. Five transgenic lines exhibited significantly greater larva growth inhibitory rates than that of DP0158 control. These results further indicate OsGRP1 plays a role in increasing ACB insect tolerance in rice compared to controls at line level.

TABLE 9
ACB assay of OsGRP1 transgenic rice under laboratory screening condition

| | Number | Number | Number of | Larvae | | | | |
|---|---|---|---|---|---|---|---|---|
| | of larva | of larvae | total | growth | CK = ZH11-TC | | CK = DP0158 | |
| | at 1^{st} | at 2^{nd} | observed | inhibitory | P | | P | |
| Line ID | instar | instar | larvae | rate (%) | value | P ≤ 0.05 | value | P ≤ 0.05 |
| DP1784 | 48 | 139 | 332 | 61.84 | 0.0044 | Y | 0.0000 | Y |
| (Construct) | | | | | | | | |
| ZH11-TC | 10 | 43 | 123 | 47.37 | | | | |
| DP0158 | 11 | 30 | 120 | 39.69 | | | | |
| DP1784.02 | 3 | 13 | 29 | 59.38 | 0.2426 | | 0.0532 | |
| DP1784.03 | 9 | 13 | 34 | 72.09 | 0.0084 | Y | 0.0007 | Y |
| DP1784.06 | 4 | 16 | 41 | 53.33 | 0.4940 | | 0.1155 | |
| DP1784.08 | 4 | 13 | 41 | 46.67 | 0.9309 | | 0.4109 | |
| DP1784.10 | 3 | 11 | 29 | 53.13 | 0.5725 | | 0.1761 | |
| DP1784.11 | 6 | 13 | 28 | 73.53 | 0.0104 | Y | 0.0013 | Y |
| DP1784.14 | 4 | 24 | 38 | 76.19 | 0.0027 | Y | 0.0002 | Y |
| DP1784.16 | 4 | 12 | 28 | 62.50 | 0.1460 | | 0.0277 | Y |
| DP1784.19 | 5 | 9 | 33 | 50.00 | 0.7955 | | 0.2672 | |
| DP1784.20 | 6 | 15 | 31 | 72.97 | 0.0097 | Y | 0.0011 | Y |

### (7) ACB Screening Results of OsAP2-4 Transgenic Rice Plants

OsAP2-4 transgenic rice plants were tested three times. All experiments showed that the average larva growth inhibitory rate of OsAP2-4 transgenic rice plants was greater than that of ZH11-TC and DP0158 controls.

In the second experiment, ten OsAP2-4 transgenic lines were placed on one 32-well plate with 6 repeats. Five days after inoculation, 676 larvae were found, 8 larvae developed to 1^{st} instar, and 300 larvae developed to 2^{nd} instar. While 76 of 193 larvae in ZH11-TC seedlings' wells developed to 2^{nd} instar; 47 of 205 larvae developed to 2^{nd} instar in DP0158 seedlings' wells. The average larva growth inhibitory rates of OsAP2-4 transgenic rice, ZH11-TC and DP0158 were 46%, 39% and 23%, respectively. The average larva growth inhibitory rate of OsAP2-4 transgenic rice was significantly greater than that of DP0158 control. These results show that over-expression of OsAP2-4 in rice significantly increased ACB insect tolerance of transgenic rice at construct level.

Further analysis at transgenic line level is displayed in Table 10. Two transgenic lines exhibited significantly greater larva growth inhibitory rates than that of ZH11-TC control. Ten transgenic lines exhibited significantly greater larva growth inhibitory rates than that of DP0158 control. These results further indicate OsAP2-4 plays a role in increasing ACB insect tolerance in rice compared to controls at line level.

TABLE 10
ACB assay of OsAP2-4 transgenic rice under laboratory screening condition

| | Number | Number | Number of | Larvae | | | | |
|---|---|---|---|---|---|---|---|---|
| | of larva | of larvae | total | growth | CK = ZH11-TC | | CK = DP0158 | |
| | at 1^{st} | at 2^{nd} | observed | inhibitory | P | | P | |
| Line ID | instar | instar | larvae | rate (%) | value | P ≤ 0.05 | value | P ≤ 0.05 |
| DP1948 | 8 | 300 | 676 | 46.20 | 0.0880 | 0.0000 | Y | |
| (Construct) | | | | | | | | |
| ZH11-TC | 0 | 76 | 193 | 39.38 | | | | |
| DP0158 | 0 | 47 | 205 | 22.93 | | | | |
| DP1948.02 | 0 | 37 | 69 | 53.62 | 0.0439 | Y | 0.0000 | Y |
| DP1948.04 | 0 | 34 | 81 | 41.98 | 0.7243 | | 0.0026 | Y |
| DP1948.05 | 1 | 23 | 68 | 36.23 | 0.6516 | | 0.0348 | Y |
| DP1948.07 | 1 | 30 | 64 | 49.23 | 0.1503 | | 0.0001 | Y |
| DP1948.08 | 1 | 28 | 69 | 42.86 | 0.5884 | | 0.0021 | Y |
| DP1948.11 | 4 | 33 | 68 | 56.94 | 0.0127 | Y | 0.0000 | Y |
| DP1948.13 | 1 | 24 | 68 | 37.68 | 0.8331 | | 0.0190 | Y |
| DP1948.14 | 0 | 25 | 53 | 47.17 | 0.2814 | | 0.0009 | Y |
| DP1948.15 | 0 | 34 | 66 | 51.52 | 0.0837 | | 0.0000 | Y |
| DP1948.19 | 0 | 32 | 70 | 45.71 | 0.3481 | | 0.0006 | Y |

### (8) ACB Screening Results of OsDUF630/DUF632 Transgenic Rice Plants

OsDUF630/DUF632 transgenic rice plants were tested two times. All experiments showed that the average larva growth inhibitory rate of OsDUF630/DUF632 transgenic rice plants was greater than that of ZH11-TC and DP0158 controls.

In the first experiment, ten OsDUF630/DUF632 transgenic lines were placed on one 32-well plate with 6 repeats. Five days after inoculation, 562 larvae were found, 10 larvae developed to 1^{st} instar, and 225 larvae developed to 2^{nd} instar. While 48 of 187 larvae in ZH11-TC seedlings' wells developed to 2^{nd} instar; 5 of 181 larvae developed to 1^{st} instar and 67 larvae developed to 2^{nd} instar in DP0158 seedlings' wells. The average larva growth inhibitory rates of OsDUF630/DUF632 transgenic rice, ZH11-TC and DP0158 were 43%, 26% and 41%, respectively. The average larva growth inhibitory rate of OsDUF630/DUF632 transgenic rice was significantly greater than that of ZH11-TC control. These results show that over-expression of OsDUF630/DUF632 in rice significantly increased ACB insect tolerance of transgenic rice at construct level.

Further analysis at transgenic line level is displayed in Table 11. Six transgenic lines exhibited significantly greater larva growth inhibitory rates than that of ZH11-TC control. One transgenic lines exhibited significantly greater larva growth inhibitory rates than that of DP0158 control. These results further indicate OsDUF630/DUF632 plays a role in increasing ACB insect tolerance in rice compared to controls at line level.

TABLE 11
ACB assay of OsDUF630/DUF632 transgenic rice under laboratory screening
condition

| | Number | Number | Number of | Larvae | | | | |
|---|---|---|---|---|---|---|---|---|
| | of larva | of larvae | total | growth | CK = ZH11-TC | | CK = DP0158 | |
| | at 1^{st} | at 2^{nd} | observed | inhibitory | P | | P | |
| Line ID | instar | instar | larvae | rate (%) | value | P ≤ 0.05 | value | P ≤ 0.05 |
| DP2583 | 10 | 225 | 562 | 42.83 | 0.0003 | Y | 0.9684 | |
| (Construct) | | | | | | | | |
| ZH11-TC | 0 | 48 | 187 | 25.67 | | | | |
| DP0158 | 5 | 67 | 181 | 41.40 | | | | |
| DP2583.03 | 2 | 30 | 62 | 53.13 | 0.0002 | Y | 0.1037 | |
| DP2583.04 | 1 | 18 | 58 | 33.90 | 0.2288 | | 0.3039 | |
| DP2583.05 | 0 | 22 | 55 | 40.00 | 0.0453 | Y | 0.8509 | |
| DP2583.06 | 0 | 35 | 59 | 59.32 | 0.0000 | Y | 0.0199 | Y |
| DP2583.07 | 0 | 9 | 43 | 20.93 | 0.5273 | | 0.0182 | |
| DP2583.09 | 0 | 21 | 54 | 38.89 | 0.0622 | | 0.7574 | |
| DP2583.10 | 2 | 27 | 60 | 50.00 | 0.0008 | Y | 0.2378 | |
| DP2583.11 | 2 | 25 | 63 | 44.62 | 0.0060 | Y | 0.6423 | |
| DP2583.13 | 1 | 17 | 51 | 36.54 | 0.1310 | | 0.5283 | |
| DP2583.16 | 2 | 21 | 57 | 42.37 | 0.0177 | Y | 0.8898 | |

Taken together, these results indicate that OsAAK1, OsDN-ITP8, OsPMR5, OsERV-B, OsbHLH065, OsGRP1, OsAP2-4, and OsDUF630/DUF632 transgenic rice plants have increased tolerance to ACB insects compared to control plants.

### Example 4

### Characterization of the Transgenic Rice Plants by OAW Assay

Oriental armyworm (OAW) was used in cross-validations of insecticidal activity. OAW belongs to Lepidoptera Noctuidae, and is a polyphagous insect pest. The eggs of OAW were obtained from the Institute of Plant Protection of Chinese Academy of Agricultural Sciences and hatched in an incubator at 27° C. The neonate larvae were used in this cross-validation assay.

Rice plants were cultured as described in Example 3, and the experiments design was similar as to ACB insect assay described in Example 3. Five days later, all the survived larvae were visually measured and given tolerant values according to Table 3.

Larvae growth inhibitory rate was used as a parameter for this insect tolerance assay, which is the percentage of the inhibited number over the statistics number of larvae, wherein the inhibited number is the sum of the tolerance value of all observed test insects from four wells in one repeat and the statistics number of larvae is the sum of the number of all the observed insects and number of larvae at 1^{st} instar.

The raw data were analyzed by Chi-square, the lines with P<0.01 were considered as OAW tolerant positive lines.

### (1) OAW Screening Results of OsAAK1 Transgenic Rice Plants

OsAAK1 transgenic rice plants were tested four times. All experiments showed that the average larva growth inhibitory rate of OsAAK1 transgenic rice plants was greater than that of DP0158 control. And two of them showed significantly greater than that of DP0158 control.

In the second experiment, ten OsAAK1 transgenic lines were placed on one 32-well plate, and repeated for 6 times. Five days after inoculation, 657 larvae were found in the OsAAK1 transgenic rice wells, wherein 201 larvae developed to 2^{nd} instar. One of the 172 larvae in the ZH11-TC wells developed to 1^{st} instar, 32 larvae developed to 2^{nd} instar; and 2 of 192 larvae in the DP0158 wells developed to 1^{st} instar and 26 developed to 2^{nd} instar. The average larva growth inhibitory rates of OsAAK1 transgenic rice, ZH11-TC and DP0158 were 31%, 20% and 15%, respectively. The average larva growth inhibitory rate of OsAAK1 transgenic rice was significantly greater than that of ZH11-TC and DP0158 controls. These results show that over-expression of OsAAK1 in rice significantly increased OAW insect tolerance of transgenic rice at construct level.

Further analysis at transgenic line level is shown in Table 12. Five lines had significantly greater larvae growth inhibitory rates than that of ZH11-TC control; seven lines have significantly greater larvae growth inhibitory rates than that of DP0158 control. These results further indicate OsAAK1 plays a role in increasing OAW insect tolerance in rice compared to controls at line level.

TABLE 12
OAW assay of OsAAK1 transgenic rice under laboratory screening condition

| | Number | Number | Number of | Larvae | | | | |
|---|---|---|---|---|---|---|---|---|
| | of larva | of larvae | total | growth | CK = ZH11-TC | | CK = DP0158 | |
| | at 1^{st} | at 2^{nd} | observed | inhibitory | P | | P | |
| Line ID | instar | instar | larvae | rate (%) | value | P ≤ 0.05 | value | P ≤ 0.05 |
| DP1931 | 0 | 201 | 657 | 30.59 | 0.0090 | Y | 0.0000 | Y |
| (Construct) | | | | | | | | |
| ZH11-TC | 1 | 32 | 172 | 19.65 | | | | |
| DP0158 | 2 | 26 | 192 | 15.46 | | | | |
| DP1931.01 | 0 | 22 | 64 | 34.38 | 0.0199 | Y | 0.0013 | Y |
| DP1931.03 | 0 | 21 | 63 | 33.33 | 0.0294 | Y | 0.0021 | Y |
| DP1931.04 | 0 | 14 | 65 | 21.54 | 0.6825 | | 0.1895 | |
| DP1931.06 | 0 | 18 | 67 | 26.87 | 0.2206 | | 0.0314 | Y |
| DP1931.10 | 0 | 25 | 68 | 36.76 | 0.0082 | Y | 0.0004 | Y |
| DP1931.11 | 0 | 20 | 68 | 29.41 | 0.1086 | | 0.0113 | Y |
| DP1931.12 | 0 | 25 | 68 | 36.76 | 0.0093 | Y | 0.0005 | Y |
| DP1931.13 | 0 | 13 | 62 | 20.97 | 0.9241 | | 0.3242 | |
| DP1931.14 | 0 | 27 | 69 | 39.13 | 0.0027 | Y | 0.0001 | Y |
| DP1931.15 | 0 | 16 | 63 | 25.40 | 0.4259 | | 0.0864 | |

### (2) OAW Screening Results of OsDN-ITP8 Transgenic Rice Plants

OsDN-ITP8 transgenic rice plants were tested three times. All experiments showed that the average larva growth inhibitory rate of OsDN-ITP8 transgenic rice plants was greater than that of ZH11-TC and DP0158 controls. And two of them showed that the average larva growth inhibitory rate of OsDN-ITP8 transgenic rice plants were significantly greater than that of DP0158 control.

In the first experiment, ten OsDN-ITP8 transgenic lines were placed on one 32-well plate, and repeated for 6 times. Four days after inoculation, 610 larvae were found in the OsDN-ITP8 transgenic rice wells, wherein 24 larvae developed to 2nd instar, 123 larvae developed to 2^{nd} instar. Nine of the 190 larvae in the ZH11-TC wells developed to 1^{st} instar, 19 larvae developed to 2^{nd} instar; and 5 of 175 larvae in the DP0158 wells developed to 1^{st} instar and 15 developed to 2^{nd} instar. The average larva growth inhibitory rates of OsDN-ITP8 transgenic rice, ZH11-TC and DP0158 were 27%, 19% and 14%, respectively. The average larva growth inhibitory rate of OsDN-ITP8 transgenic rice was significantly greater than that of ZH11-TC and DP0158 controls. These results show that over-expression of OsDN-ITP8 in rice significantly increased OAW insect tolerance of transgenic rice at construct level.

Further analysis at transgenic line level is shown in Table 13. Three lines had significantly greater larvae growth inhibitory rates than that of ZH11-TC control; seven lines have significantly greater larvae growth inhibitory rates than that of DP0158 control. These results further indicate OsDN-ITP8 plays a role in increasing OAW insect tolerance in rice compared to controls at line level.

TABLE 13
OAW assay of OsDN-ITP8 transgenic rice under laboratory screening condition

| | Number | Number | Number of | Larvae | | | | |
|---|---|---|---|---|---|---|---|---|
| | of larva | of larvae | total | growth | CK = ZH11-TC | | CK = DP0158 | |
| | at 1^{st} | at 2^{nd} | observed | inhibitory | P | | P | |
| Line ID | instar | instar | larvae | rate (%) | value | P ≤ 0.05 | value | P ≤ 0.05 |
| DP2605 | 24 | 123 | 610 | 26.97 | 0.0341 | Y | 0.0011 | Y |
| (Construct) | | | | | | | | |
| ZH11-TC | 9 | 19 | 190 | 18.59 | | | | |
| DP0158 | 5 | 15 | 175 | 13.89 | | | | |
| DP2605.01 | 1 | 11 | 57 | 22.41 | 0.5101 | | 0.1244 | |
| DP2605.02 | 1 | 14 | 59 | 26.67 | 0.1882 | | 0.0286 | Y |
| DP2605.03 | 3 | 13 | 66 | 27.54 | 0.1184 | | 0.0142 | Y |
| DP2605.04 | 4 | 9 | 61 | 26.15 | 0.1982 | | 0.0294 | Y |
| DP2605.05 | 3 | 14 | 59 | 32.26 | 0.0275 | Y | 0.0024 | Y |
| DP2605.06 | 3 | 18 | 61 | 37.50 | 0.0033 | Y | 0.0002 | Y |
| DP2605.07 | 5 | 7 | 63 | 25.00 | 0.2716 | | 0.0444 | Y |
| DP2605.08 | 0 | 8 | 62 | 12.90 | 0.3130 | | 0.8648 | |
| DP2605.09 | 3 | 17 | 61 | 35.94 | 0.0063 | Y | 0.0004 | Y |
| DP2605.10 | 1 | 12 | 61 | 22.58 | 0.5214 | | 0.1232 | |

Taken together, these results indicate that OsAAK1 and OsDN-ITP8 transgenic rice plants have increased tolerance to OAW insects compared to control plants.

1. A recombinant DNA construct comprising a polynucleotide encoding a polypeptide with amino acid sequence of at least 90% sequence identity to SEQ ID NO: 3, 6, 9, 12, 15, 18, 21 or 24 operably linked to at least one heterologous regulatory element, wherein increased expression of the polynucleotide in a plant increases insect pest tolerance.

2. The recombinant DNA construct of claim 1, wherein the polynucleotide comprises the nucleotide sequence of SEQ ID NO: 1, SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 17, SEQ ID NO: 19, SEQ ID NO: 20, SEQ ID NO: 22 or SEQ ID NO: 23.

3. The recombinant DNA construct of claim 1, wherein the encoded polypeptide comprises the amino acid sequence of SEQ ID NO: 3, SEQ ID NO: 6, SEQ ID NO: 9, SEQ ID NO: 12, SEQ ID NO: 15, SEQ ID NO: 18, SEQ ID NO: 21 or SEQ ID NO: 24.

4. The recombinant DNA construct of claim 1, wherein increased expression of the polynucleotide in a plant enhances the insect pest tolerance.

5. The recombinant DNA construct of claim 1, wherein the insect pest is a Lepidopteran.

6. The recombinant DNA construct of claim 1, wherein the insect pest is Asian Corn Borer (Ostrinia furnacalis) or Oriental Armyworm (Mythimna separata).

7. (canceled)

8. The recombinant DNA construct of claim 1, wherein the at least one heterologous regulatory element is a heterologous promoter.

9. A modified plant or seed comprising an increased expression of at least one polynucleotide encoding a polypeptide comprising an amino acid sequence of at least 90% sequence identity to SEQ ID NO: 3, 6, 9, 12, 15, 18, 21 or 24.

10. The plant of claim 9, wherein the plant comprises in its genome the recombinant DNA construct of claim 1, wherein said plant exhibits improved insect pest tolerance when compared to the control plant.

11. The plant of claim 9, wherein the plant comprises a targeted genetic modification at a genomic locus comprising a polynucleotide sequence encoding a polypeptide with an amino acid sequence of at least 90% sequence identity to SEQ ID NO: 3, 6, 9, 12, 15, 18, 21 or 24, thereby increasing expression of the polypeptide, wherein said plant exhibits improved insect pest tolerance when compared to the control plant.

12. The plant of claim 9, wherein the insect pest is a Lepidopteran.

13. The plant of claim 12, wherein the insect pest is Asian Corn Borer (Ostrinia furnacalis) or Oriental Armyworm (Mythimna separata).

14. The plant of claim 9, wherein said plant is selected from the group consisting of rice, maize, soybean, sunflower, sorghum, canola, wheat, alfalfa, cotton, barley, millet, sugar cane and switchgrass.

15. A method of increasing insect pest tolerance in a plant, comprising increasing the expression of at least one polynucleotide encoding a polypeptide comprising an amino acid sequence of at least 90% sequence identity to SEQ ID NO: 3, 6, 9, 12, 15, 18, 21 or 24.

16. The method of claim 15, wherein the method comprises:
a) expressing in a regenerable plant cell a recombinant DNA construct comprising a regulatory element operably liked to the polynucleotide sequence; and
b) generating the plant, wherein the plant comprises in its genome the recombinant DNA construct.

17. The method of claim 16, wherein the regulatory element is a heterologous promoter.

18. The method of claim 15, wherein the method comprises:
a) introducing in a regenerable plant cell a targeted genetic modification at a genomic locus that encodes the polypeptide comprising an amino acid sequence of at least 90% sequence identity compared to SEQ ID NO: 3, 6, 9, 12, 15, 18, 21 or 24; and
b) generating the plant, wherein the level and/or activity of the polypeptide is increased in the plant.

19. The method of claim 18, wherein the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), engineered site-specific meganucleases, or Argonaute.

20. (canceled)

21. The method of claim 15, wherein the insect pest is a Lepidopteran.

22. The method of claim 21, wherein the insect pest is Asian Corn Borer (Ostrinia furnacalis) or Oriental Armyworm (Mythimna separata).


SEQUENCE LISTING (40 sequences)

SEQ ID NO: 1 - DNA, 1085 bp, Oryza sativa
cgtatagccatttcgtcgaacaccttcccgagccgactgccgccgccgccgccatgctcctcgcgaagccccacctctcctcctcctctttcctcccatccacgcgggtgtctagccccgctccgggtcccaaccacgcaaagcccatcgccgcctctcccgcccctcgacgctgcctccgtctcgccgtcacatccgccgcggcgccggctgcttcgtcggcggaggcggcggcggcgctgagccgcgtggatgtgctctcagaggcgctccccttcatccagcgcttcaaggggaagaccgtggtggtgaagtacggcggcgcggcgatgaagtcgccggagctccaggcttcagtgatccgcgacctggtcctcctctcgtgcgtcggcctccaccccgtgctcgtccacggcggcggccccgagatcaactcctggctgctccgcgtcggcgtcgagccgcagttccggaacggcctccgcgtcactgacgcgctcaccatggaggtcgtcgagatggtgctcgtcggcaaggtcaacaagcacctcgtctccctcatcaacctcgcgggcggaaccgccgtaggtctctgtggcaaggacgctcgcctcctcaccgcgcgcccctccccgaacgcagcggccctcggcttcgtcggcgaggtctcgcgcgtggacgccaccgtcctccacccaatcatcgcctccggtcacatcccggtcatcgccactgtggccgccgacgagaccgggcaggcctacaacatcaacgctgacacggcggccggcgagatcgccgccgcggtcggcgcggagaagctgttgctgctcacagatgtgtctggaattctggccgaccgtaatgaccccgggagtctggtgaaagagatcgacattgctggggtgcggcagatggtggccgacgggcaggtagctggtgggatgataccgaaggtggaatgctgcgtgcgtgccctcgcacagggcgtgcacactgcaagcatcatcgatgggcgtgtcccgcactcgttgctgctcgagattctcacagatgagggcactggcactatgatcactggctga

SEQ ID NO: 2 - DNA, 1032 bp, Oryza sativa
atgctcctcgcgaagccccacctctcctcctcctctttcctcccatccacgcgggtgtctagccccgctccgggtcccaaccacgcaaagcccatcgccgcctctcccgcccctcgacgctgcctccgtctcgccgtcacatccgccgcggcgccggctgcttcgtcggcggaggcggcggcggcgctgagccgcgtggatgtgctctcagaggcgctccccttcatccagcgcttcaaggggaagaccgtggtggtgaagtacggcggcgcggcgatgaagtcgccggagctccaggcttcagtgatccgcgacctggtcctcctctcgtgcgtcggcctccaccccgtgctcgtccacggcggcggccccgagatcaactcctggctgctccgcgtcggcgtcgagccgcagttccggaacggcctccgcgtcactgacgcgctcaccatggaggtcgtcgagatggtgctcgtcggcaaggtcaacaagcacctcgtctccctcatcaacctcgcgggcggaaccgccgtaggtctctgtggcaaggacgctcgcctcctcaccgcgcgcccctccccgaacgcagcggccctcggcttcgtcggcgaggtctcgcgcgtggacgccaccgtcctccacccaatcatcgcctccggtcacatcccggtcatcgccactgtggccgccgacgagaccgggcaggcctacaacatcaacgctgacacggcggccggcgagatcgccgccgcggtcggcgcggagaagctgttgctgctcacagatgtgtctggaattctggccgaccgtaatgaccccgggagtctggtgaaagagatcgacattgctggggtgcggcagatggtggccgacgggcaggtagctggtgggatgataccgaaggtggaatgctgcgtgcgtgccctcgcacagggcgtgcacactgcaagcatcatcgatgggcgtgtcccgcactcgttgctgctcgagattctcacagatgagggcactggcactatgatcactggctga

SEQ ID NO: 3 - protein, 343 aa, Oryza sativa
MLLAKPHLSSSSFLPSTRVSSPAPGPNHAKPIAASPAPRRCLRLAVTSAAAPAASSAEAAAALSRVDVLSEALPFIQRFKGKTVVVKYGGAAMKSPELQASVIRDLVLLSCVGLHPVLVHGGGPEINSWLLRVGVEPQFRNGLRVTDALTMEVVEMVLVGKVNKHLVSLINLAGGTAVGLCGKDARLLTARPSPNAAALGFVGEVSRVDATVLHPIIASGHIPVIATVAADETGQAYNINADTAAGEIAAAVGAEKLLLLTDVSGILADRNDPGSLVKEIDIAGVRQMVADGQVAGGMIPKVECCVRALAQGVHTASIIDGRVPHSLLLEILTDEGTGTMITG

SEQ ID NO: 4 - DNA, 2472 bp, Oryza sativa
ggctaagatgtcgcagctcggcgacccggcgacgcgccctgaggaggacacttgcttcatccccacctcgtacgccattgacgaggagctgcgtgaatggagtgagaccgcagcggtctcctgggcggcccgcgctccgccgaccaccgagccgcgcgacgtcgagcaggccttcctcgatgagttcaagctccgccgtggcgaggtggccgtgtctctccatcacccacaagctttcctcatcaagttccagcaccgtcgacattgtgaggaagcgctggcgaagggatacgtcaagcggcacggcattgagatccacttcatcaagtggcgcagcctcgagagcgcgcttggtgtcgccttgatgttccgagttcggttgtgtcttgacggcgtccccatgcatgcctgggccgcggacattgctgagcgcatcattggtcgcacttgcgccctggaacagatcgagacggacgtcgtccacccagtggagtctggtaacacgcgctccatcgacctatgggcgtggacggcgaatcctagcaccatcccgaagaggatgtggcttggcttcacaaagcgggcgaaggactcgaacctggcgcccctatttgcggtggagaacccactggagcactggcagaggggggtgcgccatccggtgctcttccacttggaggagattcatgactacacggcggcgaccattgatctggaggggcaaggcagcttccagccggccaagcgctgcctaccgccttggagcctgggagtgctcgacggtgaacaggtcccagggcgagtctttgaagacttcccgcaccacccgccgccgccacgatcggtccacgagcgcctcggagggcgtgatcgggatgacgagcgccgggaggttcgccggccagaacgccggtctgaccgcgacgccatcgatggccgcgcggacagaccccgccgtggacgcgcagcacggggaggacgccttcatgatgaaggcgaccacgatgacgaggatgattgggatggcgagcgcgacgaccgcgacggccgtgctgatcgcgatggccgccatgaccgcgatcagggcgggcgcggcgggcgtggtcgtcgtcgggggtctggcaatgaccacccgagaccctggcgccgcaatgaccgcgatgatgatcgtgatgaccgaggccgggatctcggccgtggccgggatgataggcggggaagcgacaacgactaccgccgtgaacgcacgcgctccccgcgtcgccgggacaggggaggcgccaaccgtagcggcggcggcacgcggcgcacggctaatccggtctacgactcgagcaaactgatgaacattccccttctacacaaatccgcgaaccaggcagaggtaggtgaatttcgtttgctccacgcgctgcacaactccagcttcttgtgtcaggatccccagacgaacgcgttgtcgccggtgagccaactgcagcggctctccatcatcgcagacaaggctgccacgccagccctctcgggtcgcccagccaaaccgaggattgaagcctggttccaaaacgcggcctgggagccgatcccggtcgaacacgccttcgcgcgcatcaagtcggcgctgccgccgcctacctcgacgactacttgccaacaagtagaggaggcgctcctgcggattgagctggcggctgcggcgaccgacccccctgtgggagagccattcccgcagattgactcgccgcccccgcgggtcacctcgccggcattagcccaaggcccggtgctgagctctcccgtcgccacaggggggggcgtcaacttggaaatgactggtgatgcaggggggaagggcggccacctgatgctgccgccttctcccccggctgtccaggtcgtccccaccggcgcgatggagattgatgacattgtggcgccgccgccaccttccccggttgccacccagcttgcccaagcagcagcctcggcgtcaccgatctcgacgggcgtcctcgatgctctgttcgcctcgccgccgcaaccgatcatcgcctcgccgccgaggtcgccaccaagatcgccgtgtgcgcgaccgctccacatgggacgacgcctcaagatccgcacaaggcagcacagccagcccacccgacgcagtgagcgcattgccaagcaaccagcacggccgacgatggagcgctgccaacgcgttctcttcaaaaggctgggcctcctcaacggcaaggaaggcgcttcaattgagcaggtcatcgccgagtacgtcgccatgttcgatggcccactgccggcgcacgtcatcgccgccctcaccaccatcttcggcatcgacgacgaagagcaggagacaatggatgcggcgctgatatctctagttggcgaaggtatagccgacgcggcggaggatgctgaggacaccgtcgccgcctaatgcgtattcacctcggctggagt

SEQ ID NO: 5 - DNA, 2442 bp, Oryza sativa
atgtcgcagctcggcgacccggcgacgcgccctgaggaggacacttgcttcatccccacctcgtacgccattgacgaggagctgcgtgaatggagtgagaccgcagcggtctcctgggcggcccgcgctccgccgaccaccgagccgcgcgacgtcgagcaggccttcctcgatgagttcaagctccgccgtggcgaggtggccgtgtctctccatcacccacaagctttcctcatcaagttccagcaccgtcgacattgtgaggaagcgctggcgaagggatacgtcaagcggcacggcattgagatccacttcatcaagtggcgcagcctcgagagcgcgcttggtgtcgccttgatgttccgagttcggttgtgtcttgacggcgtccccatgcatgcctgggccgcggacattgctgagcgcatcattggtcgcacttgcgccctggaacagatcgagacggacgtcgtccacccagtggagtctggtaacacgcgctccatcgacctatgggcgtggacggcgaatcctagcaccatcccgaagaggatgtggcttggcttcacaaagcgggcgaaggactcgaacctggcgcccctatttgcggtggagaacccactggagcactggcagaggggggtgcgccatccggtgctcttccacttggaggagattcatgactacacggcggcgaccattgatctggaggggcaaggcagcttccagccggccaagcgctgcctaccgccttggagcctgggagtgctcgacggtgaacaggtcccagggcgagtctttgaagacttcccgcaccacccgccgccgccacgatcggtccacgagcgcctcggagggcgtgatcgggatgacgagcgccgggaggttcgccggccagaacgccggtctgaccgcgacgccatcgatggccgcgcggacagaccccgccgtggacgcgcagcacggggaggacgccttcatgatgaaggcgaccacgatgacgaggatgattgggatggcgagcgcgacgaccgcgacggccgtgctgatcgcgatggccgccatgaccgcgatcagggcgggcgcggcgggcgtggtcgtcgtcgggggtctggcaatgaccacccgagaccctggcgccgcaatgaccgcgatgatgatcgtgatgaccgaggccgggatctcggccgtggccgggatgataggcggggaagcgacaacgactaccgccgtgaacgcacgcgctccccgcgtcgccgggacaggggaggcgccaaccgtagcggcggcggcacgcggcgcacggctaatccggtctacgactcgagcaaactgatgaacattccccttctacacaaatccgcgaaccaggcagaggtaggtgaatttcgtttgctccacgcgctgcacaactccagcttcttgtgtcaggatccccagacgaacgcgttgtcgccggtgagccaactgcagcggctctccatcatcgcagacaaggctgccacgccagccctctcgggtcgcccagccaaaccgaggattgaagcctggttccaaaacgcggcctgggagccgatcccggtcgaacacgccttcgcgcgcatcaagtcggcgctgccgccgcctacctcgacgactacttgccaacaagtagaggaggcgctcctgcggattgagctggcggctgcggcgaccgacccccctgtgggagagccattcccgcagattgactcgccgcccccgcgggtcacctcgccggcattagcccaaggcccggtgctgagctctcccgtcgccacaggggggggcgtcaacttggaaatgactggtgatgcaggggggaagggcggccacctgatgctgccgccttctcccccggctgtccaggtcgtccccaccggcgcgatggagattgatgacattgtggcgccgccgccaccttccccggttgccacccagcttgcccaagcagcagcctcggcgtcaccgatctcgacgggcgtcctcgatgctctgttcgcctcgccgccgcaaccgatcatcgcctcgccgccgaggtcgccaccaagatcgccgtgtgcgcgaccgctccacatgggacgacgcctcaagatccgcacaaggcagcacagccagcccacccgacgcagtgagcgcattgccaagcaaccagcacggccgacgatggagcgctgccaacgcgttctcttcaaaaggctgggcctcctcaacggcaaggaaggcgcttcaattgagcaggtcatcgccgagtacgtcgccatgttcgatggcccactgccggcgcacgtcatcgccgccctcaccaccatcttcggcatcgacgacgaagagcaggagacaatggatgcggcgctgatatctctagttggcgaaggtatagccgacgcggcggaggatgctgaggacaccgtcgccgcctaa

SEQ ID NO: 6 - protein, 813 aa, Oryza sativa
MSQLGDPATRPEEDTCFIPTSYAIDEELREWSETAAVSWAARAPPTTEPRDVEQAFLDEFKLRRGEVAVSLHHPQAFLIKFQHRRHCEEALAKGYVKRHGIEIHFIKWRSLESALGVALMFRVRLCLDGVPMHAWAADIAERIIGRTCALEQIETDVVHPVESGNTRSIDLWAWTANPSTIPKRMWLGFTKRAKDSNLAPLFAVENPLEHWQRGVRHPVLFHLEEIHDYTAATIDLEGQGSFQPAKRCLPPWSLGVLDGEQVPGRVFEDFPHHPPPPRSVHERLGGRDRDDERREVRRPERRSDRDAIDGRADRPRRGRAARGGRLHDEGDHDDEDDWDGERDDRDGRADRDGRHDRDQGGRGGRGRRRGSGNDHPRPWRRNDRDDDRDDRGRDLGRGRDDRRGSDNDYRRERTRSPRRRDRGGANRSGGGTRRTANPVYDSSKLMNIPLLHKSANQAEVGEFRLLHALHNSSFLCQDPQTNALSPVSQLQRLSIIADKAATPALSGRPAKPRIEAWFQNAAWEPIPVEHAFARIKSALPPPTSTTTCQQVEEALLRIELAAAATDPPVGEPFPQIDSPPPRVTSPALAQGPVLSSPVATGGGVNLEMTGDAGGKGGHLMLPPSPPAVQVVPTGAMEIDDIVAPPPPSPVATQLAQAAASASPISTGVLDALFASPPQPIIASPPRSPPRSPCARPLHMGRRLKIRTRQHSQPTRRSERIAKQPARPTMERCQRVLFKRLGLLNGKEGASIEQVIAEYVAMFDGPLPAHVIAALTTIFGIDDEEQETMDAALISLVGEGIADAAEDAEDTVAA

SEQ ID NO: 7 - DNA, 1631 bp, Oryza sativa
ggactcaggccatttagctagctagccaaagccaattgccatgtggagtgctctcttctcccatctgagagaggttcacaagagaagcggagttaaggaggagaagttgataatgaagtcgccaccagcagcaggtgaggccggctgccacaagccacaggcgactgccaccaacaagatgacggtgctgcagtccccgctggggctcaggaccatcctcacctccctcgtcgccttcttcatcgtcgtcagctccgtctccctcctcttcgaccgcggccaggatgctcaggcgcaactcgccgtcgagcagcatcagcaccaagaagttctgctcaagcagaagccggcatcagcagcagtgggcgagcagaaatcagtggtagtagatcagtcgtcgttgaggagccaggaggcgcaggtgcagtggacatctgagctgcaggacgtggccacggacagcggcgacggcggcttcgacggcgaggaggactgcaactggtcgttgggacggtgggtgtacgacaacgcgtcgcggccactctactccggcttgaagtgctccttcatcttcgacgaggtggcctgcgacaagtatggaaggaatgacaccaagtaccagcactggagatggcagcctcacggttgcaaccttccaagattcaatgccacaaagtttcttgaaaagcttaggaacaagagactggtttttgtgggcgattcagtaaacagaaatcaatgggtgtcgatggtgtgcatggtggagcacttcatccctgatggccgcaagatgcgcgtttacaacggctcccttatctccttcaaagcatttgagtacaatgcgacgatagatttctactggtcaccactgctattggaatcaaacagcgacaaccccataattcacagagtggagtaccggatcataagggcagacaggattgagaaacacgccaatgtctggaaggacgctgatttcatcgtcttcaactcctacctttggtggaggaagcagagggatggtatgatgatgaaagtcatgtatggttcatttgaggacggggatgcaaagttagatgaggtgcaaatggttgatggttatgagatagctctcaagaaactaactgaatatcttggagccaatatcaacaagaacaagactagaatcttctttgcaggctcatcacctgcccattcctgggctagcaactggggaggagatgacaacaacaagtgtctaaacgaaacagaaccaattcagatagaagattataggagtgcaaccacagactacggcatgatggacaaggcgaaggagatatttggaacactggaaccaaagggcatacatgttcagatactgaacatcacccagctttctgagtaccgcaaggacgcccatccaacgatattcaggagacagtacgttcctctgacgaaagagcagattgcaaacccgagcatctacgcagactgcacgcattggtgcctccctggagttcctgatgtttggaacgagttcttgtatgcatacattatgcacaaatgatatatgtataataatgtaatcttaatttggccaaatttcttctttgttaacttgtgggtgtctaagtaggtataagtgcaca

SEQ ID NO: 8 - DNA, 1509 bp, Oryza sativa
atgtggagtgctctcttctcccatctgagagaggttcacaagagaagcggagttaaggaggagaagttgataatgaagtcgccaccagcagcaggtgaggccggctgccacaagccacaggcgactgccaccaacaagatgacggtgctgcagtccccgctggggctcaggaccatcctcacctccctcgtcgccttcttcatcgtcgtcagctccgtctccctcctcttcgaccgcggccaggatgctcaggcgcaactcgccgtcgagcagcatcagcaccaagaagttctgctcaagcagaagccggcatcagcagcagtgggcgagcagaaatcagtggtagtagatcagtcgtcgttgaggagccaggaggcgcaggtgcagtggacatctgagctgcaggacgtggccacggacagcggcgacggcggcttcgacggcgaggaggactgcaactggtcgttgggacggtgggtgtacgacaacgcgtcgcggccactctactccggcttgaagtgctccttcatcttcgacgaggtggcctgcgacaagtatggaaggaatgacaccaagtaccagcactggagatggcagcctcacggttgcaaccttccaagattcaatgccacaaagtttcttgaaaagcttaggaacaagagactggtttttgtgggcgattcagtaaacagaaatcaatgggtgtcgatggtgtgcatggtggagcacttcatccctgatggccgcaagatgcgcgtttacaacggctcccttatctccttcaaagcatttgagtacaatgcgacgatagatttctactggtcaccactgctattggaatcaaacagcgacaaccccataattcacagagtggagtaccggatcataagggcagacaggattgagaaacacgccaatgtctggaaggacgctgatttcatcgtcttcaactcctacctttggtggaggaagcagagggatggtatgatgatgaaagtcatgtatggttcatttgaggacggggatgcaaagttagatgaggtgcaaatggttgatggttatgagatagctctcaagaaactaactgaatatcttggagccaatatcaacaagaacaagactagaatcttctttgcaggctcatcacctgcccattcctgggctagcaactggggaggagatgacaacaacaagtgtctaaacgaaacagaaccaattcagatagaagattataggagtgcaaccacagactacggcatgatggacaaggcgaaggagatatttggaacactggaaccaaagggcatacatgttcagatactgaacatcacccagctttctgagtaccgcaaggacgcccatccaacgatattcaggagacagtacgttcctctgacgaaagagcagattgcaaacccgagcatctacgcagactgcacgcattggtgcctccctggagttcctgatgtttggaacgagttcttgtatgcatacattatgcacaaatga

SEQ ID NO: 9 - protein, 502 aa, Oryza sativa
MWSALFSHLREVHKRSGVKEEKLIMKSPPAAGEAGCHKPQATATNKMTVLQSPLGLRTILTSLVAFFIVVSSVSLLFDRGQDAQAQLAVEQHQHQEVLLKQKPASAAVGEQKSVVVDQSSLRSQEAQVQWTSELQDVATDSGDGGFDGEEDCNWSLGRWVYDNASRPLYSGLKCSFIFDEVACDKYGRNDTKYQHWRWQPHGCNLPRFNATKFLEKLRNKRLVFVGDSVNRNQWVSMVCMVEHFIPDGRKMRVYNGSLISFKAFEYNATIDFYWSPLLLESNSDNPIIHRVEYRIIRADRIEKHANVWKDADFIVFNSYLWWRKQRDGMMMKVMYGSFEDGDAKLDEVQMVDGYEIALKKLTEYLGANINKNKTRIFFAGSSPAHSWASNWGGDDNNKCLNETEPIQIEDYRSATTDYGMMDKAKEIFGTLEPKGIHVQILNITQLSEYRKDAHPTIFRRQYVPLTKEQIANPSIYADCTHWCLPGVPDVWNEFLYAYIMHK

SEQ ID NO: 10 - DNA, 1405 bp, Oryza sativa
tcaactctgaaacaatcaactgcaccagtgtactaattaaatatattactgtacttaagacatggctatcgcgtcgtcgtcgttttcacttgctgctatcctcctcatcatcatcatgtactgctgccccacgggtttggtggaggcagctcgcaaggggccggctgctgccggcggcggcgacgacagcgcgatgagggagaggtacgagaagtgggcggcggaccatgggcgcacgtacaaggactccctggagaaggcgcggcgattcgaggtattcaggaccaacgccctgttcatcgattcgtttaatgctgcaggaggcaagaagagcccccggctgacgaccaacaagttcgccgacctgacaaacgaagagttcgcggagtactacggtaggccgtttagcacgcctgtaattggaggtagtggcttcatgtacgggaacgtgaggccctcagacgtgccagccaacataaactggagggatagaggcgctgtcacccaagtcaagaaccaaaaggattgtggtgagctttaattattcagttcattcattcctttctttttttatatatatatgtatatcttctttttcctcctgtgtcatctcttaattaaactcaagtaactcgtaaaactaatcataataaaattaatgatcggaaaaattgcttacggatcaccagtagtactatttctatgttagatatctactagcaaatatgattttccatatattgattaactacttttttttttctttttgtgtcgtcgtatgtataaacagcgagctgttgggcgttctcagcggtggcagcggtggaaggcatccaccagatcaggagtcacaatctggttgccctctcgacgcagcaactgttggactgctccaccgggaggaacaaccatggctgcaaccgcggcgacatggacgaagcgttccgctacatcaccagcaacggcggcatcgccgccgagtcagactacccctacgaagaccgcgcgctgggcacctgccgcgcctcagggaaaccggtggcggcctccatcagaggcttccagtatgtccctccgaacaacgagaccgccctcctgctggccgtcgcccaccagcctgtgtccgtggcactcgacggcgtgggcaaggtgtcccagttcttcagcagcggagtgtttggcgcgatgcaaaatgagacgtgcaccactgacctcaaccatgccatgacggcggtggggtacggcaccgacgagcacggcaccaagtactggctaatgaagaactcgtggggaaccgactggggcgagggaggatatatgaagatcgcgcgggatgtcgcgtccaacaccggcctttgcggcctcgccatgcaaccctcttaccccgttgcctaataaactaaag

SEQ ID NO: 11 - DNA, 1074 bp, Oryza sativa
atggctatcgcgtcgtcgtcgttttcacttgctgctatcctcctcatcatcatcatgtactgctgccccacgggtttggtggaggcagctcgcaaggggccggctgctgccggcggcggcgacgacagcgcgatgagggagaggtacgagaagtgggcggcggaccatgggcgcacgtacaaggactccctggagaaggcgcggcgattcgaggtattcaggaccaacgccctgttcatcgattcgtttaatgctgcaggaggcaagaagagcccccggctgacgaccaacaagttcgccgacctgacaaacgaagagttcgcggagtactacggtaggccgtttagcacgcctgtaattggaggtagtggcttcatgtacgggaacgtgaggccctcagacgtgccagccaacataaactggagggatagaggcgctgtcacccaagtcaagaaccaaaaggattgtgcgagctgttgggcgttctcagcggtggcagcggtggaaggcatccaccagatcaggagtcacaatctggttgccctctcgacgcagcaactgttggactgctccaccgggaggaacaaccatggctgcaaccgcggcgacatggacgaagcgttccgctacatcaccagcaacggcggcatcgccgccgagtcagactacccctacgaagaccgcgcgctgggcacctgccgcgcctcagggaaaccggtggcggcctccatcagaggcttccagtatgtccctccgaacaacgagaccgccctcctgctggccgtcgcccaccagcctgtgtccgtggcactcgacggcgtgggcaaggtgtcccagttcttcagcagcggagtgtttggcgcgatgcaaaatgagacgtgcaccactgacctcaaccatgccatgacggcggtggggtacggcaccgacgagcacggcaccaagtactggctaatgaagaactcgtggggaaccgactggggcgagggaggatatatgaagatcgcgcgggatgtcgcgtccaacaccggcctttgcggcctcgccatgcaaccctcttaccccgttgcctaa

SEQ ID NO: 12 - protein, 357 aa, Oryza sativa
MAIASSSFSLAAILLIIIMYCCPTGLVEAARKGPAAAGGGDDSAMRERYEKWAADHGRTYKDSLEKARRFEVFRTNALFIDSFNAAGGKKSPRLTTNKFADLTNEEFAEYYGRPFSTPVIGGSGFMYGNVRPSDVPANINWRDRGAVTQVKNQKDCASCWAFSAVAAVEGIHQIRSHNLVALSTQQLLDCSTGRNNHGCNRGDMDEAFRYITSNGGIAAESDYPYEDRALGTCRASGKPVAASIRGFQYVPPNNETALLLAVAHQPVSVALDGVGKVSQFFSSGVFGAMQNETCTTDLNHAMTAVGYGTDEHGTKYWLMKNSWGTDWGEGGYMKIARDVASNTGLCGLAMQPSYPVA

SEQ ID NO: 13 - DNA, 494 bp, Oryza sativa
cagcagaagaggaagatgatgatgaccgaggtcgccaatcacagcaaaaggaaccacaatgaaagctacttcaccgggaaagcagcagtcaccagcagctcggaggagtttgggagcatgacatccaagaagccgaggaacacaagcccgagagacgctcccgtctccccgaaggagaagaaggataagattggtgagagagtggctgcactgcagcagctagtgtcaccatttgggaagacggacactgcttctgttcttcaggaggcctcagggtacatcaagtttcttcaccagcagctcgaggttcttagctccccttacatgcgtgctcctccggtgcctggcgctgcgcctgaggatcccgaccactacagcctgaggaaccgtggcctctgcctggttccagtggaccagacgctgcagctgacgcagagcaacggcgccgacctgtgggcgccggcgaacacgaccaggcgcaggtgaacgaggaa

SEQ ID NO: 14 - DNA, 471 bp, Oryza sativa
atgatgatgaccgaggtcgccaatcacagcaaaaggaaccacaatgaaagctacttcaccgggaaagcagcagtcaccagcagctcggaggagtttgggagcatgacatccaagaagccgaggaacacaagcccgagagacgctcccgtctccccgaaggagaagaaggataagattggtgagagagtggctgcactgcagcagctagtgtcaccatttgggaagacggacactgcttctgttcttcaggaggcctcagggtacatcaagtttcttcaccagcagctcgaggttcttagctccccttacatgcgtgctcctccggtgcctggcgctgcgcctgaggatcccgaccactacagcctgaggaaccgtggcctctgcctggttccagtggaccagacgctgcagctgacgcagagcaacggcgccgacctgtgggcgccggcgaacacgaccaggcgcaggtga

SEQ ID NO: 15 - protein, 156 aa, Oryza sativa
MMMTEVANHSKRNHNESYFTGKAAVTSSSEEFGSMTSKKPRNTSPRDAPVSPKEKKDKIGERVAALQQLVSPFGKTDTASVLQEASGYIKFLHQQLEVLSSPYMRAPPVPGAAPEDPDHYSLRNRGLCLVPVDQTLQLTQSNGADLWAPANTTRRR

SEQ ID NO: 16 - DNA, 801 bp, Oryza sativa
ttacagttcgaacattaggaagcatgagctatttccaggctacaacatacaagcctcataatgggatcattgtggacaaggtagcaataggtcttgggagtacttgcaaattgcttcatgaaagggccaaatgttcgtattccaatagattcatcaagcttcaagagcaagtatacccaaggcttcttcttgttgctgcttgccataacaggattggtcctgtgtatgcctcaagtgggaaaggaaactctgagcgtgtcaatgatcccttctccatggaatctttgaacaaagctatagctggaactaaaaagcaatggcccatacaagatatgctgatagatcaaatttctaagattagagggtctggctctggtggaaatggtggtggtaataaaaacagtcatgaaggcagtggtggtggctcagaggacgaatctttgacggagtcattatatgaaatggtccaagttttgttggcaactattgcctttatactcatgtacatccatataataagaggagaggagttataccgccttgcgagggactacactagatacctggttactggtaagagaacttccagactgaaacgcgccatgttaaactggcacaatttctgtgagggcatcaccaacaaggatagcgtgcaagagtcaacatttgaaagatcaacttctgaaccaatgtggtggcagcagcccctaaagtttgtccatcgcattgaggaactttacagaggctattttcgcccacatgcccaggaatcatagttctgatgattgaggatccctttgg

SEQ ID NO: 17 - DNA, 753 bp, Oryza sativa
atgagctatttccaggctacaacatacaagcctcataatgggatcattgtggacaaggtagcaataggtcttgggagtacttgcaaattgcttcatgaaagggccaaatgttcgtattccaatagattcatcaagcttcaagagcaagtatacccaaggcttcttcttgttgctgcttgccataacaggattggtcctgtgtatgcctcaagtgggaaaggaaactctgagcgtgtcaatgatcccttctccatggaatctttgaacaaagctatagctggaactaaaaagcaatggcccatacaagatatgctgatagatcaaatttctaagattagagggtctggctctggtggaaatggtggtggtaataaaaacagtcatgaaggcagtggtggtggctcagaggacgaatctttgacggagtcattatatgaaatggtccaagttttgttggcaactattgcctttatactcatgtacatccatataataagaggagaggagttataccgccttgcgagggactacactagatacctggttactggtaagagaacttccagactgaaacgcgccatgttaaactggcacaatttctgtgagggcatcaccaacaaggatagcgtgcaagagtcaacatttgaaagatcaacttctgaaccaatgtggtggcagcagcccctaaagtttgtccatcgcattgaggaactttacagaggctattttcgcccacatgcccaggaatcatag

SEQ ID NO: 18 - protein, 250 aa, Oryza sativa
MSYFQATTYKPHNGIIVDKVAIGLGSTCKLLHERAKCSYSNRFIKLQEQVYPRLLLVAACHNRIGPVYASSGKGNSERVNDPFSMESLNKAIAGTKKQWPIQDMLIDQISKIRGSGSGGNGGGNKNSHEGSGGGSEDESLTESLYEMVQVLLATIAFILMYIHIIRGEELYRLARDYTRYLVTGKRTSRLKRAMLNWHNFCEGITNKDSVQESTFERSTSEPMWWQQPLKFVHRIEELYRGYFRPHAQES

SEQ ID NO: 19 - DNA, 698 bp, Oryza sativa
actactccagtccacacccgcaccaccgcggcagccatggacgactcccacgacctggcctccccgacctcccctgacacggcgtcctcgtcgtcttcgtctacgtcgacatcatcgtcctccgccaccgtcgccccgaagaagcggccgcgcaacgacggccggcacccgacgtaccgcggcgtgcgcatgcggagctgggggaagtgggtgtccgagatcagggagccccgcaagaagtcgcgcatctggctgggcacgttcgccaccgcggagatggccgcgcgcgcgcacgacgtggccgcgctcgccatcaagggccgcaccgcgcacctcaacttcccggacctcgcgcacctgctcccgcgcccggccaccgcggcgcccaaggacgtgcaggcggcggcgctgctcgccgccgccgcagccgacttcccctccgtctccgtcgacgccaatgccaagagccccgacacctgctccgtcgccagcgccgcctcgccgcagccgccaccgccggacgccgaagcggaccctgacagcacgctgttcgacctcccggacctgctcctggacctgagatacgagacgtcctcgagcctctcgtgcggggcgtcgtgggccgtcgatgacgacgtggccggcggcgtcgtgttccgcctcgaggagcccatgctgtgggattactgatcatgtgg

SEQ ID NO: 20 - DNA, 654 bp, Oryza sativa
atggacgactcccacgacctggcctccccgacctcccctgacacggcgtcctcgtcgtcttcgtctacgtcgacatcatcgtcctccgccaccgtcgccccgaagaagcggccgcgcaacgacggccggcacccgacgtaccgcggcgtgcgcatgcggagctgggggaagtgggtgtccgagatcagggagccccgcaagaagtcgcgcatctggctgggcacgttcgccaccgcggagatggccgcgcgcgcgcacgacgtggccgcgctcgccatcaagggccgcaccgcgcacctcaacttcccggacctcgcgcacctgctcccgcgcccggccaccgcggcgcccaaggacgtgcaggcggcggcgctgctcgccgccgccgcagccgacttcccctccgtctccgtcgacgccaatgccaagagccccgacacctgctccgtcgccagcgccgcctcgccgcagccgccaccgccggacgccgaagcggaccctgacagcacgctgttcgacctcccggacctgctcctggacctgagatacgagacgtcctcgagcctctcgtgcggggcgtcgtgggccgtcgatgacgacgtggccggcggcgtcgtgttccgcctcgaggagcccatgctgtgggattactga

SEQ ID NO: 21 - protein, 217 aa, Oryza sativa
MDDSHDLASPTSPDTASSSSSSTSTSSSSATVAPKKRPRNDGRHPTYRGVRMRSWGKWVSEIREPRKKSRIWLGTFATAEMAARAHDVAALAIKGRTAHLNFPDLAHLLPRPATAAPKDVQAAALLAAAAADFPSVSVDANAKSPDTCSVASAASPQPPPPDAEADPDSTLFDLPDLLLDLRYETSSSLSCGASWAVDDDVAGGVVFRLEEPMLWDY

SEQ ID NO: 22 - DNA, 2909 bp, Oryza sativa
atggggtgctcgtcgtcgaagaaggtggaggaggaggcggccgtgaagacgtgccatgaccggaggagcttcgtgaagaaggcgatcgcgcagaggaacctgctcgcctcctcccatgtcgcctacgcccactccctccgccgcgtctcgctcgccctcttctactgcctcgccgaggacgagcacctctacttcctccaggacacggcggcgtcgtcggcggcgccgtgccggcaccggccgtgctcgccggagaggaaggttcttgtcatgaactggctgagaccagacgccggcggcgtcggcggcggcgcgccggtgcacccggtggtggaggtggagcagcggtgggaggagaatgatgttgccgccgagaccgtcacggtggacgggttcttcggcgcggatcccggccagctcttccacccttcgtcgtacgctccggtgaatgccatgccggcctcgccgccgccgccgcagccgacaacgacatgggacttcgtctcttgggaccctttctcctcgctccatcacgatcaccaacaatacgtgagctatggcgttgaagatgacgaagagaggaggaggagaagcgatgacgaagatgacgagcagatgccggagctggaagaagaaagcgacgacgccgccgacgacgacgacggcgacggcgatgtcaagctgcaggcggaagcttcgccggcggctgtggagcggccgatggcggaggaggaggaggaagagaagacggtggatcgcgtgaagaacgaactgagggttgtggcgagcgaggagatcgagcagcagagcacgccggggttcaccgtgtacgtggaccggccaccggcgagcatggcggaggccatgagggacatccagggccacttcgtgaagatcgtcgacaccgccaaccacgtctccgtcctcctcgaggtcgtcccctaccagaggaaaggtacacgcacgccattacagcctcttgtagttaatctctcgcaaacaatgtcttcctcttcaggaaggtttttaagtactcgatgaacaatgtctcgcatctaaactaaatagttatagaataattctaaaaattttaacaaaatagattgatgtattacactttacaaatatacaagttaaaattcaacttttacgagttataacaaaaataacaaattaaactgcaaatatagttatatataaatagagaaatatcaagtgaaaaccatcaaataaatgaaaacctgtgaaaaccatatctaaatttttgtaaattcatgaaaaaattactagagatagggacatgcatgaattacattcataccaaatatcacaaatttcgggtgaatactgttatgttactattcacacgaaatttgtcttttttttctctcaaatgaagttgagtttggacttgagatttggtatgaatgtatttcattcctgtaccaatctctagtaattttttcatgaatttataaaacttttaaatatggtttccacgggttttacttatttgatagttttgaccgaatatgtcccctatataaatataggtatactcaatttgttatttttttataacttgtaaaagttaaatttaagcttatataagtgatatattacatataaatatattttgttatttttttaaaaaataaccatttaattaacagagaagaaatattagaatccattccttcctcttcttcttctccatcattgatcattgatgtcttgtagacagtccgaccagctgctccgagcgacggtgatgacgaggaaggcggcggcgaggtctcgccggagccattcgagctcttcaagagccacaaggagagcctcgataggctctacgagtgggagaagaggctctacgaggaagtcaaggtagagaccccgatatcttccaaactctgacgtcatcgtcttcgtgtagctccgagcttttcatttcgttttccatggcggtggtggtgaaggcaggggagcgggtgaggctgtcgtacgagaggaagtgcgcgctgctgcggagccaggacgccaatggcgccgagccgtccgccatcgagaggaccagggccgccatgagagacctccgcaccaagctcgacatctccatcacctccgtcgacgccgtctccaagcggatcgccgccgtccgcgacgacgagctcctcccccagctcgcccagctcatccgagggcaagaacaatgccaatccatccatcgatctgatcttctgagatgttttttcctcttttttttgttaatttgtttgagatgttttaggttggcgaggatgtggatggtgatcgccgatgcgcaccgggtgatgaagcgcacggcggacgaggcgtgcgcgctcctctcgtcgtcgtcggcggcggcggcgcgcgcggctgcgggcggcgagggaggcgtcaggggcccgccgccgccgccggggcaggcgcgggcggccacggcggcgggcgcgctcggggcggagctccgcgggtggggcgcggcgatggaggcgtgggcggagtcgcagcgcggctacgcggcggcgctctgggggtgggcccggagctgcgtcgcggacggcgagcacatgccgcgcctcctcgccgcgtgggccgccgcggtcgaggccgtcgacgtcgaggcggccaccagggccgtggatgccctcgccgccgaggcggccgccgtcgccacggccgcgcggcggcgcggcggcgaggaggagtggaacgaggaggaggggaagaagaggatctgcgtcggcctcgcggcggcgctggcggccacggcggaggccggcggcttggcctccgccgcgtacggcgagctggtggtggagatggaagagagggagcgcgcgagggagatggcgggaagggacgaagagcaaaatcaaaactga

SEQ ID NO: 23 - DNA, 2028 bp, Oryza sativa
atggggtgctcgtcgtcgaagaaggtggaggaggaggcggccgtgaagacgtgccatgaccggaggagcttcgtgaagaaggcgatcgcgcagaggaacctgctcgcctcctcccatgtcgcctacgcccactccctccgccgcgtctcgctcgccctcttctactgcctcgccgaggacgagcacctctacttcctccaggacacggcggcgtcgtcggcggcgccgtgccggcaccggccgtgctcgccggagaggaaggttcttgtcatgaactggctgagaccagacgccggcggcgtcggcggcggcgcgccggtgcacccggtggtggaggtggagcagcggtgggaggagaatgatgttgccgccgagaccgtcacggtggacgggttcttcggcgcggatcccggccagctcttccacccttcgtcgtacgctccggtgaatgccatgccggcctcgccgccgccgccgcagccgacaacgacatgggacttcgtctcttgggaccctttctcctcgctccatcacgatcaccaacaatacgtgagctatggcgttgaagatgacgaagagaggaggaggagaagcgatgacgaagatgacgagcagatgccggagctggaagaagaaagcgacgacgccgccgacgacgacgacggcgacggcgatgtcaagctgcaggcggaagcttcgccggcggctgtggagcggccgatggcggaggaggaggaggaagagaagacggtggatcgcgtgaagaacgaactgagggttgtggcgagcgaggagatcgagcagcagagcacgccggggttcaccgtgtacgtggaccggccaccggcgagcatggcggaggccatgagggacatccagggccacttcgtgaagatcgtcgacaccgccaaccacgtctccgtcctcctcgaggtcgtcccctaccagaggaaagtccgaccagctgctccgagcgacggtgatgacgaggaaggcggcggcgaggtctcgccggagccattcgagctcttcaagagccacaaggagagcctcgataggctctacgagtgggagaagaggctctacgaggaagtcaaggcaggggagcgggtgaggctgtcgtacgagaggaagtgcgcgctgctgcggagccaggacgccaatggcgccgagccgtccgccatcgagaggaccagggccgccatgagagacctccgcaccaagctcgacatctccatcacctccgtcgacgccgtctccaagcggatcgccgccgtccgcgacgacgagctcctcccccagctcgcccagctcatccgagggcaagaacaatgccaatccatccatcgatctgatcttctgagatgttttttcctcttttttttgttaatttgtttgagatgttttaggttggcgaggatgtggatggtgatcgccgatgcgcaccgggtgatgaagcgcacggcggacgaggcgtgcgcgctcctctcgtcgtcgtcggcggcggcggcgcgcgcggctgcgggcggcgagggaggcgtcaggggcccgccgccgccgccggggcaggcgcgggcggccacggcggcgggcgcgctcggggcggagctccgcgggtggggcgcggcgatggaggcgtgggcggagtcgcagcgcggctacgcggcggcgctctgggggtgggcccggagctgcgtcgcggacggcgagcacatgccgcgcctcctcgccgcgtgggccgccgcggtcgaggccgtcgacgtcgaggcggccaccagggccgtggatgccctcgccgccgaggcggccgccgtcgccacggccgcgcggcggcgcggcggcgaggaggagtggaacgaggaggaggggaagaagaggatctgcgtcggcctcgcggcggcgctggcggccacggcggaggccggcggcttggcctccgccgcgtacggcgagctggtggtggagatggaagagagggagcgcgcgagggagatggcgggaagggacgaagagcaaaatcaaaactga

SEQ ID NO: 24 - protein, 675 aa, Oryza sativa
MGCSSSKKVEEEAAVKTCHDRRSFVKKAIAQRNLLASSHVAYAHSLRRVSLALFYCLAEDEHLYFLQDTAASSAAPCRHRPCSPERKVLVMNWLRPDAGGVGGGAPVHPVVEVEQRWEENDVAAETVTVDGFFGADPGQLFHPSSYAPVNAMPASPPPPQPTTTWDFVSWDPFSSLHHDHQQYVSYGVEDDEERRRRSDDEDDEQMPELEEESDDAADDDDGDGDVKLQAEASPAAVERPMAEEEEEEKTVDRVKNELRVVASEEIEQQSTPGFTVYVDRPPASMAEAMRDIQGHFVKIVDTANHVSVLLEVVPYQRKVRPAAPSDGDDEEGGGEVSPEPFELFKSHKESLDRLYEWEKRLYEEVKAGERVRLSYERKCALLRSQDANGAEPSAIERTRAAMRDLRTKLDISITSVDAVSKRIAAVRDDELLPQLAQLIRGQEQCQSIHRSDLLRCFFLFFLLICLRCFRLARMWMVIADAHRVMKRTADEACALLSSSSAAAARAAAGGEGGVRGPPPPPGQARAATAAGALGAELRGWGAAMEAWAESQRGYAAALWGWARSCVADGEHMPRLLAAWAAAVEAVDVEAATRAVDALAAEAAAVATAARRRGGEEEWNEEEGKKRICVGLAAALAATAEAGGLASAAYGELVVEMEERERAREMAGRDEEQNQN

SEQ ID NO: 25 - DNA, 24 bp, Artificial Sequence
  Forward primer for cloning cDNA of OsAAK1 gene
cgtatagccatttcgtcgaacacc

SEQ ID NO: 26 - DNA, 24 bp, Artificial Sequence
  Reverse primer for cloning cDNA of OsAAK1 gene
tcagccagtgatcatagtgccagt

SEQ ID NO: 27 - DNA, 29 bp, Artificial Sequence
  Forward primer for cloning gDNA of OsDN-ITP8 gene
ctgctgaggggctaagatgtcgcagctcg

SEQ ID NO: 28 - DNA, 31 bp, Artificial Sequence
  Reverse primer for cloning gDNA of OsDN-ITP8 gene
ccgctgaggactccagccgaggtgaatacgc

SEQ ID NO: 29 - DNA, 25 bp, Artificial Sequence
  Forward primer for cloning cDNA of OsPMR5 gene
ggactcaggccatttagctagctag

SEQ ID NO: 30 - DNA, 27 bp, Artificial Sequence
  Reverse primer for cloning cDNA of OsPMR5 gene
tgtgcacttatacctacttagacaccc

SEQ ID NO: 31 - DNA, 35 bp, Artificial Sequence
  Forward primer for cloning gDNA of OsERV-B gene
ctgctgaggtcaactctgaaacaatcaactgcacc

SEQ ID NO: 32 - DNA, 36 bp, Artificial Sequence
  Reverse primer for cloning gDNA of OsERV-B gene
ccgctgaggctttagtttattaggcaacggggtaag

SEQ ID NO: 33 - DNA, 35 bp, Artificial Sequence
  Forward primer for cloning cDNA of OsbHLH065 gene
ctgctgaggcagcagaagaggaagatgatgatgac

SEQ ID NO: 34 - DNA, 31 bp, Artificial Sequence
  Reverse primer for cloning cDNA of OsbHLH065 gene
ccgctgaggttcctccgttcacctgcgcctg

SEQ ID NO: 35 - DNA, 35 bp, Artificial Sequence
  Forward primer for cloning cDNA of OsGRP1 gene
ctgctgaggttacagttcgaacattaggaagcatg

SEQ ID NO: 36 - DNA, 35 bp, Artificial Sequence
  Reverse primer for cloning cDNA of OsGRP1 gene
ccgctgaggccaaagggatcctcaatcatcagaac

SEQ ID NO: 37 - DNA, 23 bp, Artificial Sequence
  Forward primer for cloning cDNA of OsAP2-4 gene
actactccagtccacacccgcac

SEQ ID NO: 38 - DNA, 25 bp, Artificial Sequence
  Reverse primer for cloning cDNA of OsAP2-4 gene
ccacattgatcagtaatcccacagc

SEQ ID NO: 39 - DNA, 30 bp, Artificial Sequence
  Forward primer for cloning gDNA of OsDUF630/DUF632 gene
ctgctgaggatggggtgctcgtcgtcgaag

SEQ ID NO: 40 - DNA, 34 bp, Artificial Sequence
  Reverse primer for cloning gDNA of OsDUF630/DUF632 gene
ccgctgaggtcagttttgattttgctcttcgtcc